(19) United States
(12) Patent Application Publication
(10) Pub. No.: US 2026/0130902 A1
(43) Pub. Date: May 14, 2026

(54) HIPK4 Degraders as Non-Hormonal Male Contraceptives

(71) Applicants: The Board of Trustees of the Leland Stanford Junior University, Stanford, CA (US); Sanford Burnham Prebys Medical Discovery Institute, La Jolla, CA (US)

(72) Inventors: James Kenneth Chen, Stanford, CA (US); Zaile Zhuang, Stanford, CA (US); Thomas D.Y. Chung, Del Mar, CA (US); Patrick C. Kearney, La Jolla, CA (US); Ian Pass, San Diego, CA (US); Andrey Bobkov, Escondido, CA (US); Shubhankar Dutta, San Diego, CA (US)

(21) Appl. No.: 19/380,687

(22) Filed: Nov. 5, 2025

Related U.S. Application Data
(60) Provisional application No. 63/718,510, filed on Nov. 8, 2024.

(51) Int. Cl. A61K 31/4706; A61K 31/4709; A61K 31/496; C07D 215/54; C07D 401/12; C07D 405/12; C07D 417/12

(52) U.S. Cl. CPC A61K 31/4706; A61K 31/4709; A61K 31/496; C07D 215/54; C07D 401/12; C07D 405/12; C07D 417/12

(57) ABSTRACT

Disclosed herein are compounds that abrogate the activity of HIPK4 by inhibiting the enzyme, inducing its degradation, or both. Also disclosed are methods for using the disclosed compounds as male contraceptives.

### CROSS REFERENCE TO RELATED APPLICATION

Pursuant to 35 U.S.C. § 119(e), this application claims priority to the filing date of U.S. Provisional Patent Application No. 63/718,510, filed Nov. 8, 2024, the disclosure of which is incorporated herein by reference in its entirety.

### ACKNOWLEDGEMENT OF GOVERNMENT SUPPORT

This invention was made with Government support under contract GM127030 and HD099720 awarded by the National Institutes of Health. The Government has certain rights in the invention.

### INTRODUCTION

Since the introduction of “the Pill” in the 1950s, there have been gender disparities in contraceptive autonomy and responsibility. While a variety of hormonal therapies and barrier methods have been developed for female contraception, birth control options for men have been largely restricted to condoms and vasectomy. Efforts to develop orally available and reversible male contraceptives still await success. Hormonal male contraceptives are limited by their ability to cause metabolic derangements, mood alterations, thrombotic events, acne, and testicular atrophy. Non-hormonal agents in development include inhibitors of retinoic acid signaling, bromodomain testis-specific protein, soluble adenylate cyclase, and serine/threonine kinase. However, the clinical utility of these drugs may be restricted by the somatic functions of their targets or closely related isoforms. Meanwhile, it is estimated that 85 million unintended pregnancies occur globally each year. Accordingly there remains an unmet need for safe, reversible male contraceptives.

### SUMMARY

Disclosed herein are compounds that abrogate the activity of HIPK4 by inhibiting the enzyme, inducing its degradation, or both. Also disclosed are methods for using the disclosed compounds as male contraceptives.

In one aspect herein disclosed HIPK4 inhibitors and degrader compounds have the formula:

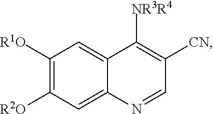

wherein R¹, R², R³, and R⁴ are as described herein.

### BRIEF DESCRIPTION OF THE FIGURES

FIG. 1 illustrates the selectivity of inhibitor Compound 1 (left-hand panel) and inhibitor Compound 22 (right-hand panel) in a 96-kinase panel, demonstrating that kinome-wide specificity is improved by altering quinoline substituents.

FIG. 2 illustrates HIPK4 activity (as a percentage of control) versus Compound 1 concentration in an ADP-Glo assay at two different ATP concentrations, demonstrating HIPK4 inhibition in an ATP-competitive manner.

FIG. 3 illustrates HIPK activity (as a percentage of control) versus Compound 1 concentration in ADP-Glo assays, demonstrating the selectivity of the inhibitor for HIPK4 over other HIPK family members.

FIG. 4A provides a comparison of wild-type, K40S, and Y175F HIPK4 activities in SHH-LIGHT2 cells and western blots of the FLAG-tagged proteins and Hedgehog pathway target GLI1.

FIG. 4B provides a graph showing inhibition of Hipk4-induced Gli reporter activity in SHH-LIGHT2 cells by a Type II HIPK4 inhibitor.

FIG. 4C illustrates a BRET assay for active site-binding inhibitors of HIPK4.

FIG. 4D provides a graph of BRET signal versus inhibitor concentration demonstrating suppression of HIPK4-luciferase BRET activity in live cells by a Type II HIPK4 inhibitor.

FIG. 5A illustrates a HiBiT-based chemiluminescent assay of targeted protein degradation (POI—protein of interest).

FIG. 5B illustrates a hypothetical protein degradation time course profile annotated with key metrics of PROTAC efficacy.

FIG. 5C provides a graph of chemiluminescence observed in LgBiT-expressing immortalized cells transiently transfected with a HIPK4-HiBiT expression construct.

FIG. 6A illustrates the structure of a CRBN-recruiting PROTAC compound.

FIG. 6B illustrates luminescence (Relative Light Units) versus time (hours) demonstrating HIPK4 degradation by the compound of FIG. 6A.

FIG. 7A illustrates the structure of a CRBN-recruiting PROTAC compound.

FIG. 7B illustrates luminescence (Relative Light Units) versus time (hours) demonstrating HIPK4 degradation by the compound of FIG. 7A.

FIG. 8 shows a gel stained with anti-HIPK4 demonstrating degradation of HiBiT-HIPK4 transiently expressed in immortalized cells by a quinoline compound with an estimated DC₅₀ (half maximal degradation concentration) of 45 nM.

FIG. 9 provides a graph of HiBiT-HIPK4 construct signal versus time in the presence of a HIPK4 inhibitor disclosed herein in a whole-cell assay, demonstrating HIPK4 degradation.

FIG. 10 provides a graph of HiBiT-HIPK4 construct signal versus time in the presence of a HIPK4 inhibitor disclosed herein in a whole-cell assay, demonstrating HIPK4 degradation.

FIG. 11 provides a graph of HiBiT-HIPK4 construct signal versus time in the presence of a HIPK4 inhibitor disclosed herein in a whole-cell assay, demonstrating HIPK4 degradation.

FIG. 12 provides a graph of HiBiT-HIPK4 construct signal versus time in the presence of HIPK4 inhibitor Compound 26 disclosed herein in a whole-cell assay, demonstrating HIPK4 degradation.

FIG. 13 provides a graph of HiBiT-HIPK4 construct signal versus time in the presence of a HIPK4 inhibitor disclosed herein in a whole-cell assay, demonstrating HIPK4 degradation.

FIG. 14 provides a graph of HiBiT-HIPK4 construct signal versus time in the presence of a HIPK4 inhibitor disclosed herein in a whole-cell assay, demonstrating HIPK4 degradation.

FIG. 15 provides a graph of HiBiT-HIPK4 construct signal versus time in the presence of a quinoline analog disclosed herein having low HIPK4 inhibitory activity.

FIG. 16 provides a graph of HiBiT-HIPK4 construct signal versus time in the presence of promiscuous kinase inhibitor and potent HIPK4 inhibitor pacritinib for comparison.

FIG. 17A illustrates a HiBiT- and BRET-based assay to detect ternary complex formation between a target protein, a PROTAC compound and an E3 ligase.

FIG. 17B illustrates a HiBiT- and BRET-based assay to detect polyubiquitination of a target protein.

FIG. 18 provides structures for exemplary quinoline compounds disclosed herein.

FIG. 19 provides structures and HIPK4 IC₅₀ values for exemplary quinolines.

FIG. 20 provides structures and HIPK4 IC₅₀ values for exemplary 3-anilino quinolines.

FIG. 21 provides structures and HIPK4 IC₅₀ values for exemplary 3-alkyamino quinolines.

### DETAILED DESCRIPTION

Disclosed herein are compounds that abrogate the activity of HIPK4 by inhibiting the enzyme, inducing its degradation, or both. Also disclosed are methods for using the disclosed compounds as male contraceptives.

The present inventors have identified and characterized homeodomain-interacting kinase 4 (HIPK4) for non-hormonal male contraception (Crapster, J. A., Rack, P. G., Hellmann, Z. J., Le, A. D., Adams, C. M., Leib, R. D., Elias, J. E., Perrino, J., Behr, B., Li, Y., Lin, J., Zeng, H., and Chen, J. K. (2020) HIPK4 is essential for murine spermiogenesis. Elife 9). HIPK4 is selectively expressed in differentiating spermatids, and Hipk4 knockout male mice are infertile but otherwise exhibit normal development, physiology, and behavior. Deleterious HIPK4 mutations have also been discovered in men with non-obstructive azoospermia. Accordingly, disclosed herein are compounds and methods for male contraception via blocking HIPK4 function, removing HIPK4, or both.

In one embodiment the disclosed compounds bind to HIPK4. In certain such embodiments, the disclosed compounds inhibit the kinase activity of HIPK4. The particular mode of HIPK4 binding by the disclosed HIPK4 inhibitors may be different for different inhibitors. However, in one embodiment, the HIPK4 inhibitors comprise an inhibitor of a kinase activity of HIPK4. Such inhibitors may be Type I, Type II, Type III, Type IV, Type V and/or Type VI HIPK4 inhibitors. In certain embodiments, HIPK4 inhibitors disclosed herein are Type I and/or Type II inhibitors of HIPK4. In another embodiment, HIPK4 inhibitors disclosed herein are Type III inhibitors of HIPK4.

In one aspect of the disclosure, the compounds are bifunctional molecules, both inhibiting the kinase activity of HIPK4 and inducing the degradation of HIPK4. Certain bifunctional molecules disclosed herein include conjugates of a HIPK4-binding moiety or ligand and a ligase recruiting ligand. In one embodiment such conjugate molecules are referred to herein as proteolysis targeting chimera (PROTAC) molecules.

### HIPK4 Ligands:

As defined herein the terms HIPK4 binder, HIPK4 modulator and HIPK4 ligand are used interchangeably and describe a compound that binds to, modulates or is a ligand for HIPK4. A HIPK4 inhibitor is a HIPK4 ligand that modulates HIPK4 by inhibiting the kinase activity of HIPK4.

In certain embodiments, a HIPK4 ligand both inhibits the kinase activity of HIPK4 and induces its degradation.

In one embodiment, HIPK4 ligands disclosed herein have formula (I):

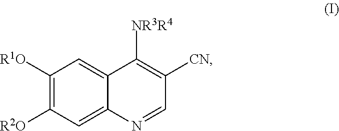

wherein
R¹ is independently selected from hydrogen, —X-L^{A}-W, and C₁₋₆ alkyl optionally substituted with one or more R^{a}, R^{b}, or both;
R² is independently selected from hydrogen, —X-L^{B}-W, and C₁₋₆ alkyl optionally substituted with one or more of the same or different R^{a}, R^{b}, or both;
X is, for each occurrence, independently selected from a covalent bond, —C(O)—, —C(O)O—, —C(O)N(R^{d})—, and —S(O)₂—;
L^{A} is a linker;
L^{B} is a linker;
W is a E3 ubiquitin ligase binding moiety;
R³ and R⁴ are independently selected from hydrogen, C₁₋₆ alkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl, each optionally substituted with one or more of the same or different R^{a}, R^{b}, or both, or R³ and R⁴ are taken together with the nitrogen atom to which they are bonded to form a 5 to 8-membered cycloheteroalkyl or heteroaryl which may optionally include one or more of the same or different additional heteroatoms and which may optionally be substituted with one or more of the same or different which may optionally be substituted with one or more of the same or different R^{e} groups;
each R^{a} is independently selected from hydrogen, C₁₋₈ alkyl, C₂₋₆ alkenyl, C₂₋₆ alkynyl, C₁₋₆ haloalkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl;
R^{b} is independently selected from the group consisting of ═O, —OR^{a}, halogen, C₁₋₃haloalkyloxy, —OCF₃, ═S, —SR^{d}, ═NR^{d}, ═NOR^{d}, —NR^{c}R^{c}, —SF₅,
   halogen, —CF₃, —CN, —NO₂, —S(O)R^{d}, —S(O)₂R^{d}, —S(O)₂OR^{d},
   —N(H)S(O)₂R^{d}, —S(O)NR^{c}R^{c}, —S(O)₂NR^{c}R^{c}, —OS(O)R^{d}, —OS(O)₂R^{d},
   —OS(O)₂OR^{d}, —OS(O)₂NR^{c}R^{c}, —C(O)R^{d}, —C(O)OR^{d}, —C(O)NR^{c}R^{c},
   —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙ NR^{c}R^{c} and —[NR^{a}C(NR^{a})]ₙNR^{c}R^{c};
each R^{c} independently is hydrogen, R^{a}, or, alternatively, two R^{c} are taken together with the nitrogen atom to which they are bonded to form a 5 to 10-membered heterocyclylalkyl which may optionally include 1, 2 or 3 additional heteroatoms selected from O, N and S, and may optionally be substituted with one or more of the same or different R^{e} groups;
each R^{d} is independently hydrogen or C₁₋₆ alkyl;
each R^{e} is independently R^{d}, ═O, —OR^{a}, halogen, —C(O)R^{d}, and —S(O)₂R^{d}; and
each n is independently an integer from 0 to 3.

In one embodiment of the disclosed HIPK4 ligands of formula (I), R¹ is C₁₋₆ alkyl optionally substituted with one or more R^{a}, R^{b}, or both. In one embodiment, R² is C₁₋₆ alkyl optionally substituted with one or more of the same or different R^{a}, R^{b}, or both. In certain such compounds, R¹ is unsubstituted C₁₋₆ alkyl. In one aspect of the disclosed HIPK4 ligands, R² may be an unsubstituted C₁₋₆ alkyl. In certain embodiments, both R¹ and R² are unsubstituted C₁₋₆ alkyl, such as the same or different unsubstituted C₁₋₆ alkyl. By way of example, R¹ and R² may be independently selected from such unsubstituted C₁₋₆ alkyl groups as methyl, ethyl, propyl, isopropyl, n-butyl, isobutyl, sec-butyl, t-butyl, n-pentyl, and neopentyl.

In one embodiment of compounds of formula (I), one of R³ and R⁴ is hydrogen. In one embodiment, one of R³ and R⁴ is hydrogen, and the other is selected from hydrogen, C₁₋₆ alkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl, each optionally substituted with one or more of the same or different R^{a}, R^{b}, or both, or R³ and R⁴ are taken together with the nitrogen atom to which they are bonded to form a 5 to 8-membered cycloheteroalkyl or heteroaryl which may optionally include one or more of the same or different additional heteroatoms and which may optionally be substituted with one or more of the same or different which may optionally be substituted with one or more of the same or different R^{e} groups.

In one embodiment of formula (I), HIPK4 ligands disclosed herein have one of R³ and R⁴ as hydrogen, and the other selected from C₁₋₆ alkyl, C₃₋₈ cycloalkyl, C₆₋₁₀ aryl, C₅₋₁₀ heteroaryl, C₆₋₁₆ arylalkyl, each optionally substituted. In one such embodiment, one of R³ and R⁴ is hydrogen, and the other is optionally substituted C₁₋₆ alkyl. For example, certain HIPK4 ligands have formula (I) wherein one of R³ and R⁴ is hydrogen, and the other is unsubstituted C₁₋₆ alkyl. In other embodiments of HIPK4 ligands of formula (I), one of R³ and R⁴ is hydrogen, and the other is C₁₋₆ alkyl substituted with one or more of the same or different R^{a}, R^{b}, or both. In one embodiment of formula (I), wherein one of R³ and R⁴ is hydrogen, and the other is C₁₋₆ alkyl, the C₁₋₆ alkyl, is sec-butyl.

In one embodiment of formula (I), one of R³ and R⁴ is hydrogen, and the other is substituted or unsubstituted C₃₋₈ cycloalkyl. In one aspect of such embodiments, one of R³ and R⁴ is hydrogen, and the other is C₃₋₈ cycloalkyl substituted with one or more of the same or different R^{a}, R^{b}, or both. In another aspect of compounds one of R³ and R⁴ is hydrogen, and the other is substituted or unsubstituted C₃₋₈ cycloalkyl, the C₃₋₈ cycloalkyl is unsubstituted.

In further embodiments, one of R³ and R⁴ is hydrogen, and the other is optionally substituted C₆₋₁₀ aryl. In one embodiment, one of R³ and R⁴ is hydrogen, and the other is optionally substituted phenyl. In one embodiment of formula (I), one of R³ and R⁴ is hydrogen, and the other is unsubstituted phenyl. In another embodiment of formula (I), one of R³ and R⁴ is hydrogen, and the other is phenyl substituted with one or more of the same or different R^{a}, R^{b}, or both, such as wherein the phenyl is substituted with 1, 2 or 3 R^{b} groups. In one aspect of such an embodiment of formula (I), the phenyl is substituted with halogen, such as chloro. In another aspect of such an embodiment of formula (I), the phenyl is substituted with one or more —OR^{a}, such as wherein the phenyl is substituted with one, two or three —OCH₃.

In one embodiment of formula (I), one of R³ and R⁴ is hydrogen, and the other is optionally substituted C₅₋₁₀ heteroaryl. In one aspect of such embodiments, one of R³ and R⁴ is hydrogen, and the other is unsubstituted C₅₋₁₀ heteroaryl. In one aspect of embodiments wherein one of R³ and R⁴ is hydrogen, and the other is optionally substituted C₅₋₁₀ heteroaryl, the C₅₋₁₀ heteroaryl is a bicyclic heteroaryl.

In one embodiment of formula (I), R³ and R⁴ are taken together with the nitrogen atom to which they are bonded to form a 5 to 8-membered cycloheteroalkyl or heteroaryl which may optionally include one or more of the same or different additional heteroatoms and which may optionally be substituted with one or more of the same or different which may optionally be substituted with one or more of the same or different R^{e} groups. For example, in certain embodiments, R³ and R⁴ are taken together with the nitrogen atom to which they are bonded to form a five-membered ring, such as a pyrrolidinyl ring, or a six-membered ring, such as a piperidinyl, piperazinyl, or morpholinyl ring.

### HIPK4 Ligand—E3 Ligase Ligand Conjugates:

Also disclosed herein are bifunctional molecules comprising a conjugate of a HIPK4-binding moiety or ligand and a ligase recruiting ligand. In one embodiment such conjugate molecules are referred to herein as proteolysis targeting chimera (PROTAC) molecules, or “PROTACs.” With continued reference to formula (I), such conjugates disclosed herein include those wherein at least one of R¹ and R² comprise an E3 ubiquitin ligase binding moiety.

In one embodiment of conjugates according to formula (I), both of R¹ and R² comprise an E3 ubiquitin ligase binding moiety. Such conjugate compounds can be represented by formula (Ia):

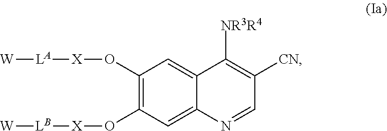

wherein
X is, for each occurrence, a conjugation moiety independently selected from a covalent bond, —C(O)—, —C(O)O—, —C(O)N(R^{d})—, and —S(O)₂—;
L^{A} is a linker;
L^{B} is a linker; and
W is, for each occurrence, independently the same or different E3 ubiquitin ligase binding moiety.

In other embodiments, conjugates herein have formula (I), wherein one of R¹ and R² comprises an E3 ubiquitin ligase binding moiety. In such embodiments of formula (I), when R¹ is hydrogen or C₁₋₆ alkyl optionally substituted with one or more R^{a}, R^{b}, or both, R² is -L^{B}-W, and when R² is hydrogen or C₁₋₆ alkyl optionally substituted with one or more R^{a}, R^{b}, or both, R¹ is -L^{A}-W;
L^{A} is a linker;
L^{B} is a linker; and
W is a E3 ubiquitin ligase binding moiety. Such conjugate compounds disclosed herein can, for example, be represented by formulas (Ib) or (Ic):

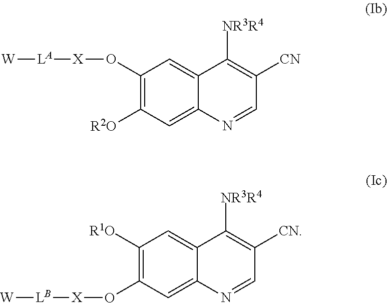

In certain embodiments of formulas (I), (Ia), (Ib) and (Ic), X represents a covalent bond. Such conjugate compounds disclosed herein can, for example, be represented by formulas (Id), (Ie), or (If):

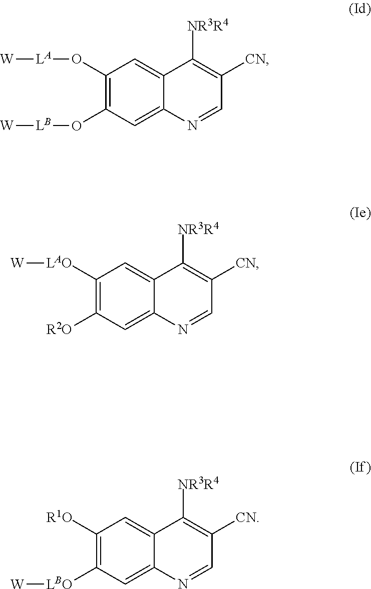

Any convenient groups may be utilized for the linkers L^{A} and L^{B} in the subject conjugates of formula (I). In certain embodiments, the linker L^{A} and L^{B} may include a group selected from alkyl, substituted alkyl, alkenyl, substituted alkenyl, alkynyl, substituted alkynyl, alkoxy, substituted alkoxy, amino, substituted amino, carboxyl, carboxyl ester, acyl amino, alkylamide, substituted alkylamide, aryl, substituted aryl, heteroaryl, substituted heteroaryl, cycloalkyl, substituted cycloalkyl, heterocyclyl, and substituted heterocyclyl. In certain embodiments, the linker L^{A}, L^{B}, or both, include an alkyl or substituted alkyl group. In certain embodiments, the linker L^{A}, L^{B}, or both, include an alkenyl or substituted alkenyl group. In certain embodiments, the L^{A}, L^{B}, or both, include an alkynyl or substituted alkynyl group. In certain embodiments, the linker L^{A}, L^{B}, or both, include an alkoxy or substituted alkoxy group. In certain embodiments, the linker L^{A}, L^{B}, or both, include an amino or substituted amino group. In certain embodiments, the linker L^{A}, L^{B}, or both, include a carboxyl or carboxyl ester group. In certain embodiments, the linker L^{A}, L^{B}, or both, include an acyl amino group. In certain embodiments, the linker L^{A}, L^{B}, or both, include an alkylamide or substituted alkylamide group. In certain embodiments, the linker L^{A} may include an aryl or substituted aryl group. In certain embodiments, the linker L^{A}, L^{B}, or both, include a heteroaryl or substituted heteroaryl group. In certain embodiments, the linker L^{A}, L^{B}, or both, include a cycloalkyl or substituted cycloalkyl group. In certain embodiments, the linker L^{A}, L^{B}, or both, include a heterocyclyl or substituted heterocyclyl group.

In some embodiments of formula (I), L^{A}, L^{B}, or both, are a linker (e.g., a first linker) described by the formula:

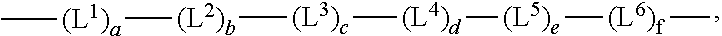

wherein L¹, L², L³, L⁴, L⁵ and L⁶ are each independently a linker subunit, and a, b, c, d, e and f are each independently 0 or 1, wherein the sum of a, b, c, d, e and f is 1 to 6.

In certain embodiments of formula (I), the sum of a, b, c, d, e and f is 1. In certain embodiments, the sum of a, b, c, d, e and f is 2. In certain embodiments, the sum of a, b, c, d, e and f is 3. In certain embodiments, the sum of a, b, c, d, e and f is 4. In certain embodiments, the sum of a, b, c, d, e and f is 5. In certain embodiments, the sum of a, b, c, d, e and f is 6. In certain embodiments, a, b, c, d, e and f are each 1. In certain embodiments, a, b, c, d and e are each 1 and f is 0. In certain embodiments, a, b, c and d are each 1 and e and f are each 0. In certain embodiments, a, b, and c are each 1 and d, e and f are each 0. In certain embodiments, a and b are each 1 and c, d, e and f are each 0. In certain embodiments, a is 1 and b, c, d, e and f are each 0.

In certain embodiments, the linker L^{A}, L^{B}, or both, include a polymer. For example, the polymer may include a polyalkylene glycol and derivatives thereof, including polyethylene glycol, methoxypolyethylene glycol, polyethylene glycol homopolymers, polypropylene glycol homopolymers, copolymers of ethylene glycol with propylene glycol (e.g., where the homopolymers and copolymers are unsubstituted or substituted at one end with an alkyl group), polyvinyl alcohol, polyvinyl ethyl ethers, polyvinylpyrrolidone, combinations thereof, and the like. In certain embodiments, the polymer is a polyalkylene glycol. In certain embodiments, the polymer is a polyethylene glycol. Other linkers are also possible, as shown in the conjugates and compounds described in more detail below.

Any convenient linker subunits L¹, L², L³, L⁴, L⁵ and L⁶ may be utilized in the linkers L^{A} and L^{B}. Linker subunits of interest include, but are not limited to, units of polymers such as polyethylene glycols, polyethylenes and polyacrylates, amino acid residue(s), carbohydrate-based polymers or carbohydrate residues and derivatives thereof, polynucleotides, alkyl groups, aryl groups, heterocyclic groups, combinations thereof, and substituted versions thereof. In some embodiments, each of L¹, L², L³, L⁴, L⁵ and L⁶ (if present) comprise one or more groups independently selected from a polyethylene glycol, a modified polyethylene glycol, an amino acid residue, an alkyl group, a substituted alkyl, an aryl group, a substituted aryl group, and a diamine (e.g., a linking group that includes an alkylene diamine).

In some embodiments, L^{A}, L^{B}, or both are a linker comprising -(L¹)ₐ-(L²)_{b}-(L³)_{c}-(L⁴)_{d}-(L⁵)ₑ-(L⁶)_{f}-, where:

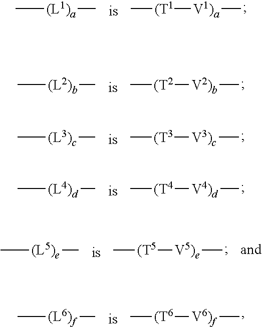

wherein T¹, T², T³, T⁴, T⁵ and T⁶, if present, are tether groups;
V¹, V², V³, V⁴, V⁵ and V⁶, if present, are covalent bonds or linking functional groups; and
a, b, c, d, e and f are each independently 0 or 1, wherein the sum of a, b, c, d, e and f is 1, 2, 3, 4, 5 or 6, such as 1, 2 or 3.

As described above, in certain embodiments, L¹ is attached to the conjugation moiety, X (e.g., as illustrated in formula (Ia) above). As such, in certain embodiments, T¹ is attached to the conjugation moiety, X (e.g., as shown in formula (Ia) above).

Thus, with reference to formulas (I), (Ia), (Ib), (Id) and (Ie), L^{A} may have the formula:

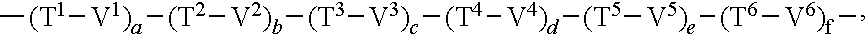

wherein
a, b, c, d, e and f are each independently 0 or 1;
T¹, T², T³, T⁴, T⁵ and T⁶ are each independently selected from a covalent bond, (C₁-C₁₂)alkylene, substituted (C₁-C₁₂)alkylene, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(C(R^{a})(OH))ₘ—, 4-amino-piperidine (4AP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n in the group (PEG)ₙ is an integer from 1 to 30, each p is an integer from 1 to 20, and each m in the group —(C(R^{a})(OH))ₘ— is an integer from 1 to 12;
V¹, V², V³, V⁴, V⁵ and V⁶ are each independently selected from the group consisting of a covalent bond, —C(O)—, —(CH₂)_{q}—C(O)—, —C(O)—(CH₂)_{q}—, —NR¹⁵—, —NR¹⁵(CH₂)_{q}—, —NR¹⁵(C₆H₄)—, —(CH₂)_{q}—C(O)NR¹⁵—, —C(O)N(R¹⁵)—, —C(O)N(R¹⁵)—(CH₂)_{q}—, —NR¹⁵C(O)—, —(CH₂)_{q}—NR¹⁵C(O)—, —C(O)O—, —(CH₂)_{q}—C(O)O—, —OC(O)—, —OC(O)—(CH₂)_{q}—, —O—, —S—, —S(O)—, —SO₂—, —SO₂N(R¹⁵)—, —(CH₂)_{q}—SO₂N(R¹⁵)—, —NR¹⁵SO₂— and —P(O)OH—, wherein each q is an integer from 1 to 6; and
each R¹⁵ is independently selected from R^{a} and —C(O)R^{a}.

With reference to formulas (I), (Ia), (Ic), (Id) and (If), -L^{B}- may be represented by the

formula:

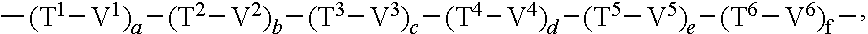

wherein
a, b, c, d, e and f are each independently 0 or 1;
T¹, T², T³, T⁴, T⁵ and T⁶ are each independently selected from a covalent bond, (C₁-C₁₂)alkylene, substituted (C₁-C₁₂)alkylene, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(C(R^{a})(OH))ₘ—, 4-amino-piperidine (4AP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each m is an integer from 1 to 12;
V¹, V², V³, V⁴, V⁵ and V⁶ are each independently selected from the group consisting of a covalent bond, —C(O)—, —(CH₂)_{q}—C(O)—, —C(O)—(CH₂)_{q}—, —NR¹⁵—, —NR¹⁵(CH₂)_{q}—, —NR¹⁵(C₆H₄)—, —(CH₂)_{q}—C(O)NR¹⁵—, —C(O)N(R¹⁵)—, —C(O)N(R¹⁵)—(CH₂)_{q}—, —NR¹⁵C(O)—, —(CH₂)_{q}—NR¹⁵C(O)—, —C(O)O—, —(CH₂)_{q}—C(O)O—, —OC(O)—, —OC(O)—(CH₂)_{q}—, —O—, —S—, —S(O)—, —SO₂—, —SO₂N(R¹⁵)—, —(CH₂)_{q}—SO₂N(R¹⁵)—, —NR¹⁵SO₂— and —P(O)OH—, wherein each q is an integer from 1 to 6; and
each R¹⁵ is independently selected from R^{a} and —C(O)R^{a}.

By way of illustration using Formula (Ib), an embodiment wherein the sum of a, b, c, d, e and f is 1, the conjugate may be represented by formula (Ib1):

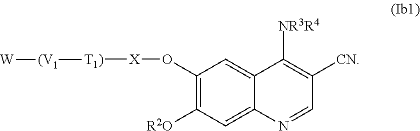

Likewise, in an embodiment of Formula (Ic), wherein the sum of a, b, c, d, e and f is 1, the conjugate may be represented by formula (Ic1):

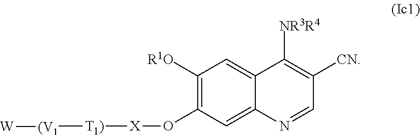

Regarding the tether groups, T¹, T², T³, T⁴, T⁵ and T⁶, any convenient tether groups may be utilized in the subject linkers. In some embodiments, T¹, T², T³, T⁴, T⁵ and T⁶ each comprise one or more groups independently selected from a covalent bond, a (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₘ—, 4-amino-piperidine (4AP), an acetal group, a hydrazine, a disulfide, and an ester, where each w is an integer from 1 to 20, each n is an integer from 1 to 30, each p is an integer from 1 to 20, and each m is an integer from 1 to 12.

In certain embodiments, the tether group (e.g., T¹, T², T³, T⁴, T⁵ and/or T⁶) includes a (C₁-C₁₂)alkyl or a substituted (C₁-C₁₂)alkyl. In certain embodiments, (C₁-C₁₂)alkyl is a straight chain or branched alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, (C₁-C₁₂)alkyl may be an alkyl or substituted alkyl, such as C₁-C₁₂ alkyl, or C₁-C₁₀ alkyl, or C₁-C₆ alkyl, or C₁-C₃ alkyl. In some instances, (C₁-C₁₂)alkyl is a C₂-alkyl. For example, (C₁-C₁₂)alkyl may be an alkylene or substituted alkylene, such as C₁-C₁₂ alkylene, or C₁-C₁₀ alkylene, or C₁-C₆ alkylene, or C₁-C₃ alkylene. In some instances, (C₁-C₁₂)alkyl is a C₂-alkylene (e.g., CH₂CH₂). In some instances, (C₁-C₁₂)alkyl is a C₃-alkylene (e.g., CH₂CH₂CH₂).

In certain embodiments, the tether group (e.g., T¹, T², T³, T⁴, T⁵ and/or T⁶) includes (C₁-C₁₂)alkyl or a substituted (C₁-C₁₂)alkyl.

In certain embodiments wherein the tether group is substituted (C₁-C₁₂)alkyl, the substituted (C₁-C₁₂)alkyl is a straight chain or branched substituted alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, substituted (C₁-C₁₂)alkyl may be a substituted alkyl, such as substituted C₁-C₁₂ alkyl, or substituted C₁-C₁₀ alkyl, or substituted C₁-C₆ alkyl, or substituted C₁-C₃ alkyl. In some instances, substituted (C₁-C₁₂)alkyl is a substituted C₂-alkyl. For example, substituted (C₁-C₁₂)alkyl may be a substituted alkylene, such as substituted C₁-C₁₂ alkylene, or substituted C₁-C₁₀ alkylene, or substituted C₁-C₆ alkylene, or substituted C₁-C₃ alkylene. In some instances, substituted (C₁-C₁₂)alkyl is a substituted C₂-alkylene. In some instances, substituted (C₁-C₁₂)alkyl is a substituted C₃-alkylene. For example, substituted (C₁-C₁₂)alkyl may include C₁-C₁₂ alkylene (e.g., C₃-alkylene or C₅-alkylene) substituted with a (PEG)ₙ group as described herein (e.g., —CONH(PEG)₃ or —NHCO(PEG)₇).

In certain embodiments, the tether group (e.g., T¹, T², T³, T⁴, T⁵ and/or T⁶) includes an ethylene diamine (EDA) moiety, e.g., an EDA containing tether group. In certain embodiments, (EDA)_{w} includes one or more EDA moieties, such as where w is an integer from 1 to 50, such as from 1 to 40, from 1 to 30, from 1 to 20, from 1 to 12 or from 1 to 6, such as 1, 2, 3, 4, 5 or 6). The linked ethylene diamine (EDA) moieties may optionally be substituted at one or more convenient positions with any convenient substituents, e.g., with an alkyl, a substituted alkyl, an acyl, a substituted acyl, an aryl or a substituted aryl. In certain embodiments, the EDA moiety is described by the structure:

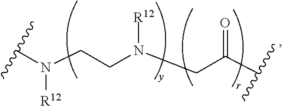

where y is an integer from 1 to 6, or is 0 or 1, and each R¹² is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, y is 1, 2, 3, 4, 5 or 6. In certain embodiments, y is 1 and r is 0. In certain embodiments, y is 1 and r is 1. In certain embodiments, y is 2 and r is 0. In certain embodiments, y is 2 and r is 1. In certain embodiments, each R¹² is independently selected from hydrogen, an alkyl, a substituted alkyl, an aryl and a substituted aryl. In certain embodiments, any two adjacent R¹² groups of the EDA may be cyclically linked, e.g., to form a piperazinyl ring. In certain embodiments, y is 1 and the two adjacent R¹² groups are an alkyl group, cyclically linked to form a piperazinyl ring. In certain embodiments, y is 1 and the adjacent R¹² groups are selected from hydrogen, an alkyl (e.g., methyl) and a substituted alkyl (e.g., lower alkyl-OH, such as ethyl-OH or propyl-OH).

In certain embodiments, the tether group (e.g., T¹, T², T³, T⁴, T⁵ and/or T⁶) includes a 4-amino-piperidine (4AP) moiety (also referred to herein as piperidin-4-amino, P4A). The 4AP moiety may optionally be substituted at one or more convenient positions with any convenient substituents, e.g., with an alkyl, a substituted alkyl, a polyethylene glycol moiety, an acyl, a substituted acyl, an aryl or a substituted aryl. In certain embodiments, the 4AP moiety is described by the structure:

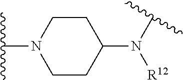

where R¹² is selected from hydrogen, alkyl, substituted alkyl, a polyethylene glycol moiety (e.g., a polyethylene glycol or a modified polyethylene glycol), alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R¹² is a polyethylene glycol moiety. In certain embodiments, R¹² is a carboxy modified polyethylene glycol.

In certain embodiments, R¹² includes an ethylene glycol, oligoethylene glycol, or polyethylene glycol moiety described by the formula: (PEG)ₖ, which may be represented by the structure:

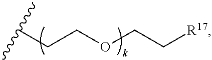

where k is an integer from 1 to 20, such as from 1 to 18, or from 1 to 16, or from 1 to 14, or from 1 to 12, or from 1 to 10, or from 1 to 8, or from 1 to 6, or from 1 to 4, or 1 or 2, such as 1, 2, 3, 4, 5, 6, 7, 8, 9, 10, 11, 12, 13, 14, 15, 16, 17, 18, 19 or 20. In some instances, k is 2. In certain embodiments, R¹⁷ is selected from OH, OR, COOH, or COOR, where R is selected from alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl. In certain embodiments, R¹⁷ is COOH. In certain embodiments, R¹⁷ is OH. In certain embodiments, R¹⁷ is OR, such as OCH₃.

In certain embodiments, a tether group (e.g., T¹, T², T³, T⁴, T⁵ and/or T⁶) includes (PEG)ₙ, where (PEG)ₙ is an ethylene glycol, oligoethylene glycol, polyethylene glycol or a modified polyethylene glycol linking unit. In certain embodiments, (PEG)ₙ is described by the structure:

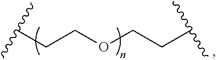

where n is an integer from 1 to 50, such as from 1 to 40, from 1 to 30, from 1 to 20, from 1 to 12 or from 1 to 6, such as 1, 2, 3, 4, 5 or 6. In some instances of formula (I) herein, n is 2. In some instances, n is 3. In some instances, n is 6. In some instances, n is 12.

In certain embodiments, a tether group (e.g., T¹, T², T³, T⁴, T⁵ and/or T⁶) includes (AA)ₚ, where AA is an amino acid residue and p is an integer from 1 to 20. Any convenient amino acid (AA) may be utilized. Amino acids of interest include but are not limited to, L- and D-amino acids, naturally occurring amino acids such as any of the 20 primary alpha-amino acids and beta-alanine, non-naturally occurring amino acids (e.g., amino acid analogs), such as a non-naturally occurring alpha-amino acid or a non-naturally occurring beta-amino acid. In certain embodiments, p is an integer from 1 to 12 or from 1 to 6, such as 1, 2, 3, 4, 5, 6, 7, 8, 9, 10, 11 or 12. In certain embodiments of formula (I), p is 1. In other certain embodiments, p is 2.

In certain embodiments, a tether group (e.g., T¹, T², T³, T⁴, T⁵ and/or T⁶) includes a moiety described by the formula —(CR¹³OH)ₘ—, where m is 0 or n is an integer from 1 to 20, such as from 1 to 12 or from 1 to 6, such as 1, 2, 3, 4, 5 or 6. In certain embodiments of formula (I), m is 1. In certain other embodiments, m is 2. In certain embodiments, R¹³ is selected from hydrogen, alkyl, substituted alkyl, aryl, and substituted aryl.

Regarding the linking functional groups, V¹, V², V³, V⁴, V⁵ and V⁶, any convenient linking functional groups may be utilized in the linker. Linking functional groups of interest include, but are not limited to, amino, carbonyl, amido, oxycarbonyl, carboxy, sulfonyl, sulfoxide, sulfonylamino, aminosulfonyl, thio, oxy, phospho, phosphoramidate, thiophosphoridate, and the like. In some embodiments, V¹, V², V³, V⁴, V⁵ and V⁶ are each independently selected from the group consisting of a covalent bond, —C(O)—, —(CH₂)_{q}—C(O)—, —C(O)—(CH₂)_{q}—, —NR¹⁵—, —NR¹⁵(CH₂)_{q}—, —NR¹⁵(C₆H₄)—, —(CH₂)_{q}—C(O)NR¹⁵—, —C(O)N(R¹⁵)—, —C(O)N(R¹⁵)—(CH₂)_{q}—, —NR¹⁵C(O)—, —(CH₂)_{q}—NR¹⁵C(O)—, —C(O)O—, —(CH₂)_{q}—C(O)O—, —OC(O)—, —OC(O)—(CH₂)_{q}—, —O—, —S—, —S(O)—, —SO₂—, —SO₂N(R¹⁵)—, —(CH₂)_{q}—SO₂N(R¹⁵)—, —NR¹⁵SO₂— and —P(O)OH—, wherein each q is an integer from 1 to 6. In certain embodiments, q is an integer from 1 to 6 (e.g., 1, 2, 3, 4, 5 or 6). In certain embodiments, q is 1. In certain embodiments, q is 2. In certain embodiments, q is 3. In certain embodiments, q is 4. In certain embodiments, q is 5. In certain embodiments, q is 6.

In some embodiments, each R¹⁵ is independently selected from R^{a} and —C(O)R^{a}.

In certain embodiments, R¹⁵ is hydrogen. In certain embodiments, each R¹⁵ is hydrogen. In certain embodiments, R¹⁵ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl.

As described above, in some embodiments, L^{A} is a linker comprising -(T¹-V¹)ₐ-(T²-V²)_{b}-(T³-V³)_{c}-(T⁴-V⁴)_{d}-(T⁵-V⁵)ₑ-(T⁶-V⁶)_{f}-, where a, b, c, d, e and f are each independently 0 or 1, where the sum of a, b, c, d, e and f is 1 to 6.

In some embodiments, in the linker L^{A}, L^{B}, or both:
T¹ is selected from (PEG)ₙ, wherein n is 1 or 2;
V¹ is —NR¹⁵—;
R¹⁵ is hydrogen; and
b, c, d, e and f are each 0.

In some embodiments, in the linker L^{A}, L^{B}, or both:
a and b are each 1, and c, d, e and f are each 0;
T¹ is selected from (PEG)ₙ, wherein n is 1 or 2;
V¹ is —C(O)N(R¹⁵)—;
R¹⁵ is hydrogen;
T² is (C₁-C₁₂)alkyl; and
V² is —O—.

With reference to formulas (I), (Ia), (Ib), (Ib1), (Ic), (Ic1), (Id), (Ie) and (If), W is, for each occurrence, independently selected from E3 ubiquitin ligase binding moieties. Exemplary E3 ligases targeted by the presently disclosed conjugate molecules include, without limitation, CRBN, VHL, IAP, MDM2, FEM1B, RNF114, and combinations thereof. Accordingly, in the presently disclosed conjugates, W may be a ligand for CRBN, VHL, IAP, MDM2, FEM1B, RNF114, or a combination thereof.

In one embodiment, the ligase binding moiety, W, binds to a ligase that is expressed in spermatids, and in certain embodiments to a ligase that is more abundantly expressed in spermatids than other tissues. By way of example, such ligases include the spermatid-selective family members FEM-1 homology B (FEM1B) and ring finger protein 114 (RNF114). Accordingly, in certain embodiments, the conjugates of formulas (I), (Ia), (Ib) and (Ic) comprise a W moiety that binds to FEM1B), RNF114, or both.

Thus, conjugates of formulas (I), (Ia), (Ib) and (Ic) may comprise a W moiety that is a FEM1B ligand. In certain examples, wherein W comprises a FEM1B ligand, W has the structure:

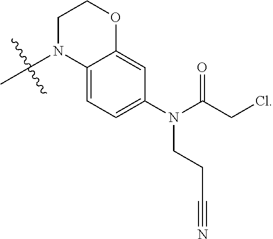 [SMILES: *N1CCOc2cc(N(CCC#N)C(=O)CCl)ccc21]

In certain embodiments, conjugates of formulas (I), (Ia), (Ib) and (Ic) may comprise a W moiety that is an RNF114 ligand. In certain examples wherein W comprises an RNF114 ligand, W has the structure:

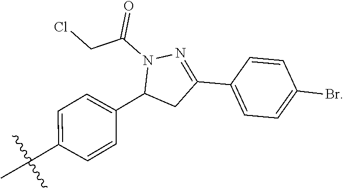 [SMILES: *c1ccc(C2CC(c3ccc(Br)cc3)=NN2C(=O)CCl)cc1]

In certain embodiments of formulas (I), (Ia), (Ib) and (Ic), W is a CRBN ligand. In certain such embodiments, exemplary W moieties that bind to CRBN have a structure selected from:

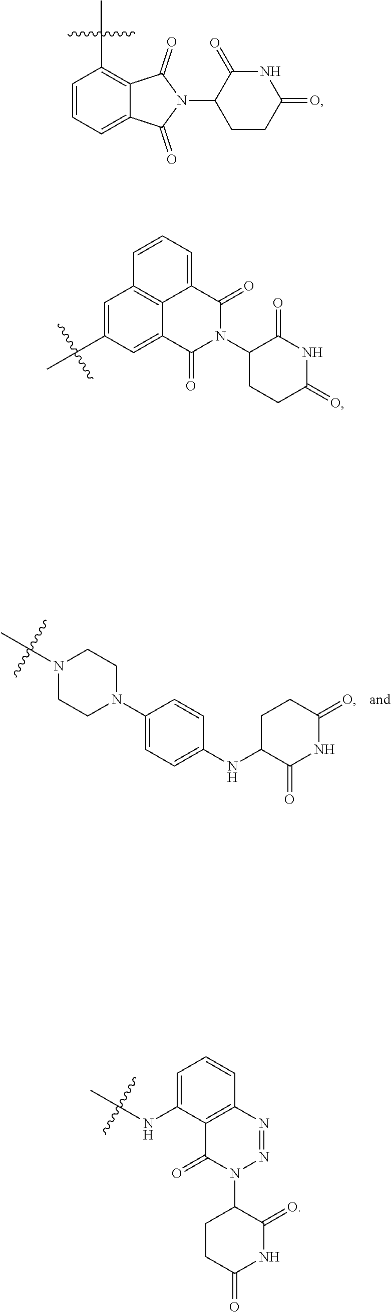 [SMILES: *c1cccc2c1C(=O)N(C1CCC(=O)NC1=O)C2=O ; *c1cc2c3c(cccc3c1)C(=O)N(C1CCC(=O)NC1=O)C2=O ; *N1CCN(c2ccc(NC3CCC(=O)NC3=O)cc2)CC1 ; "and" ; *Nc1cccc2nnn(C3CCC(=O)NC3=O)c(=O)c12]

In certain embodiments of formulas (I), (Ia), (Ib) and (Ic), W is a VHL ligand. By way of example, a W moiety used in exemplary compounds that binds to VHL has the structure:

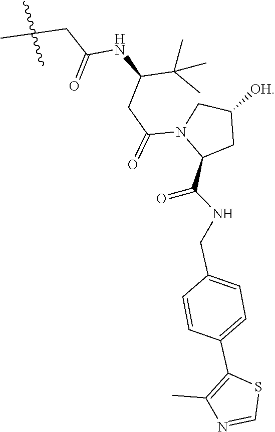 [SMILES: *CC(=O)N[C@H](CC(=O)N1C[C@H](O)C[C@H]1C(=O)NCc1ccc(-c2scnc2C)cc1)C(C)(C)C]

R³ and R⁴ in formulas (Ia), (Ib) and (Ic) are the same as described with respect to formula (I). For example, in one embodiment of compounds of formulas (I), (Ia), (Ib) and (Ic), one of R³ and R⁴ is hydrogen. In one such embodiment, one of R³ and R⁴ is hydrogen, and the other is selected from hydrogen, C₁₋₆ alkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl, each optionally substituted with one or more of the same or different R^{a}, R^{b}, or both, or R³ and R⁴ are taken together with the nitrogen atom to which they are bonded to form a 5 to 8-membered cycloheteroalkyl or heteroaryl which may optionally include one or more of the same or different additional heteroatoms and which may optionally be substituted with one or more of the same or different which may optionally be substituted with one or more of the same or different R^{e} groups.

In one embodiment of formulas (I), (Ia), (Ib) and (Ic), HIPK4 ligands disclosed herein have one of R³ and R⁴ as hydrogen, and the other selected from C₁₋₆ alkyl, C₃₋₈ cycloalkyl, C₆₋₁₀ aryl, C₅₋₁₀ heteroaryl, C₆₋₁₆ arylalkyl, each optionally substituted. In one such embodiment, one of R³ and R⁴ is hydrogen, and the other is optionally substituted C₁₋₆ alkyl. For example, certain HIPK4 ligands have formula (I), (Ia), (Ib) and/or (Ic), wherein one of R³ and R⁴ is hydrogen, and the other is unsubstituted C₁₋₆ alkyl. In other embodiments of HIPK4 ligands of formula (I), (Ia), (Ib) and/or (Ic), one of R³ and R⁴ is hydrogen, and the other is C₁₋₆ alkyl substituted with one or more of the same or different R^{a}, R^{b}, or both. In one embodiment of formula (I), wherein one of R³ and R⁴ is hydrogen, and the other is C₁₋₆ alkyl, the C₁₋₆ alkyl, is sec-butyl.

In one embodiment of formulas (I), (Ia), (Ib) and (Ic), one of R³ and R⁴ is hydrogen, and the other is substituted or unsubstituted C₃₋₈ cycloalkyl. In one aspect of such embodiments, one of R³ and R⁴ is hydrogen, and the other is C₃₋₈ cycloalkyl substituted with one or more of the same or different R^{a}, R^{b}, or both. In another aspect of compounds one of R³ and R⁴ is hydrogen, and the other is substituted or unsubstituted C₃₋₈ cycloalkyl, the C₃₋₈ cycloalkyl is unsubstituted.

In further embodiments, one of R³ and R⁴ is hydrogen, and the other is optionally substituted C₆₋₁₀ aryl. In one embodiment, one of R³ and R⁴ is hydrogen, and the other is optionally substituted phenyl. In one embodiment of formula (I), one of R³ and R⁴ is hydrogen, and the other is unsubstituted phenyl. In another embodiment of formulas (Ia), (Ib) and (Ic), one of R³ and R⁴ is hydrogen, and the other is phenyl substituted with one or more of the same or different R^{a}, R^{b}, or both, such as wherein the phenyl is substituted with 1, 2 or 3 R^{b} groups. In one aspect of such an embodiment of formulas (I), (Ia), (Ib) and (Ic), the phenyl is substituted with halogen, such as chloro. In another aspect of such an embodiment of formulas (I), (Ia), (Ib) and (Ic), the phenyl is substituted with one or more —OR^{a}, such as wherein the phenyl is substituted with one, two or three —OCH₃.

In one embodiment of formulas (I), (Ia), (Ib) and/or (Ic), one of R³ and R⁴ is hydrogen, and the other is optionally substituted C₅₋₁₀ heteroaryl. In one aspect of such embodiments, one of R³ and R⁴ is hydrogen, and the other is unsubstituted C₅-heteroaryl. In one aspect of embodiments wherein one of R³ and R⁴ is hydrogen, and the other is optionally substituted C₅₋₁₀ heteroaryl, the C₅₋₁₀ heteroaryl is a bicyclic heteroaryl.

In one embodiment of formulas (I), (Ia), (Ib) and/or (Ic), R³ and R⁴ are taken together with the nitrogen atom to which they are bonded to form a 5 to 8-membered cycloheteroalkyl or heteroaryl which may optionally include one or more of the same or different additional heteroatoms and which may optionally be substituted with one or more of the same or different which may optionally be substituted with one or more of the same or different R^{e} groups. For example, in certain embodiments, R³ and R⁴ are taken together with the nitrogen atom to which they are bonded to form a five-membered ring, such as a pyrrolidinyl ring, or a six-membered ring, such as a piperidinyl, piperazinyl, or morpholinyl ring.

### Formulations of HIPK4 Ligands and Ligand Conjugates:

The compounds of the present disclosure, including ligands and ligand conjugates of formulas (I), (Ia), (Ib), and/or (Ic), can be formulated in a variety of different ways. In general, the compound is formulated in a manner compatible with the compound, including, where applicable, the HIPK4-binding ligand and the ligase recruiting ligand, the condition to be treated, and the route of administration to be used.

In certain embodiments, a present compound is administered to the subject orally, transdermally, or by injection. In related embodiments, the compound is administered in the form of a tablet or capsule. In related embodiments, the inhibitor is administered by parenteral injection, intramuscular injection, intravenous injection, subcutaneous implantation, subcutaneous injection, or transdermal preparation. Accordingly, the present compounds are formulated in compositions suitable for such modes of administration. For example, in some embodiments, provided is a pharmaceutical composition that includes any of the compounds of the present disclosure and a pharmaceutically acceptable excipient.

The compound (e.g., a compound of formulas (I), (Ia), (Ib), and/or (Ic),) can be provided in any suitable form, e.g., in the form of a pharmaceutically acceptable salt, and can be formulated for any suitable route of administration, e.g., oral, topical or parenteral administration. Where the compound is provided as a liquid injectable (such as in those embodiments where the compound is administered intravenously or directly into a tissue), the compound can be provided as a ready-to-use dosage form, or as a reconstitutable storage-stable powder or liquid composed of pharmaceutically acceptable carriers and excipients.

Methods for formulating compounds can be adapted from those readily available. For example, compounds can be provided in a pharmaceutical composition comprising a therapeutically effective amount of a compound and a pharmaceutically acceptable carrier (e.g., saline). The pharmaceutical composition may optionally include other additives (e.g., buffers, stabilizers, preservatives, and the like). In some embodiments, the formulations are suitable for administration to a mammal, such as those that are suitable for administration to a human subject.

### Use of HIPK4 Ligands and Ligand Conjugates:

The present disclosure provides compounds and methods for blocking the activity of HIPK4 in a subject in need thereof. In one embodiment, inhibiting HIPK4 includes inhibiting the enzymatic activity of HIPK4, inducing the degradation of the enzyme, or both.

Without limitation to any particular theory, in one embodiment, the compounds recruit an E3 ligase to HIPK4, thereby inducing degradation of HIPK4 via the ubiquitination pathway. By way of example, such compounds, HIPK4 degraders, include both HIPK4 binders, such as HIPK4 inhibitors, as well as PROTACs, which are conjugates of HIPK4-binding molecules with an E3 ligase binding moiety. Accordingly, in one aspect of the present disclosure compounds that bind both HIPK4 and an E3 ligase are provided.

The present disclosure also relates to using the compounds and compositions described herein as male contraceptives. Without limitation to any particular theory, the present compounds, in one embodiment, block spermiogenesis and therefore male fertility. Thus, the presently disclosed compounds represent a new class of safe and reversible male contraceptives.

In one aspect, the present disclosure provides methods for reducing or inhibiting spermatozoa emission involving administering an effective amount of a HIPK4 inhibitor to a male subject. In embodiments, the inhibitor is a compound having a formula delineated herein, such as a compound of formula (I), a derivative thereof, or a pharmaceutically acceptable salt thereof.

In embodiments, the presently disclosed methods involve administering a compound of formula (I) in an amount sufficient to suppress spermatogenesis. In certain embodiments, the methods involve administering the inhibitor in an amount sufficient to induce azoospermia or oligozoospermia.

In embodiments, the methods involve administering the inhibitor in an amount sufficient to lower the spermatozoa concentration to not more than 3 million/mL, 2 million/mL, 1 million/mL, 0.5 million/mL, 0.25 million/mL, or 0.1 million/mL. In related embodiments, the methods involve administering the inhibitor in an amount sufficient to lower the spermatozoa concentration to not more than 0.1 million/mL.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

### Exemplary Terms

“Alkyl” refers to monovalent saturated aliphatic hydrocarbyl groups having from 1 to 10 carbon atoms (C₁₋₁₀ alkyl) and such as 1 to 6 carbon atoms (C₁₋₆ alkyl), or 1 to 5 (C₁₋₅ alkyl), or 1 to 4 (C₁₋₄alkyl), or 1 to 3 (C₁₋₃ alkyl) carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain (except the C₁ carbon atom) have been optionally replaced.

“Alkylene” refers to divalent aliphatic hydrocarbyl groups preferably having from 1 to 6 and more preferably 1 to 3 carbon atoms that are either straight-chained or branched, and which are optionally interrupted with one or more groups selected from —O—, —NR¹⁰—, —NR¹⁰C(O)—, —C(O)NR¹⁰— and the like, wherein R¹⁰ is selected from R^{d}, —C(O)R^{d}, and —S(O)₂R^{d}. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

“Substituted alkylene” refers to an alkylene group having from 1 to 3 hydrogens replaced with substituents as described for carbons in the definition of “substituted” below.

The term “alkylaminoalkyl”, “alkylaminoalkenyl” and “alkylaminoalkynyl” refers to the groups R′NHR″— where R′ is alkyl group as defined herein and R″ is alkylene, alkenylene or alkynylene group as defined herein.

The term “alkaryl” or “aralkyl” refers to the groups -alkylene-aryl and -substituted alkylene-aryl where alkylene, substituted alkylene and aryl are defined herein.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “haloalkoxy” refers to the groups alkyl-O— wherein one or more hydrogen atoms on the alkyl group have been substituted with a halo group and include, by way of examples, groups such as trifluoromethoxy, and the like.

The term “haloalkyl” refers to a substituted alkyl group as described above, wherein one or more hydrogen atoms on the alkyl group have been substituted with a halo group. Examples of such groups include, without limitation, fluoroalkyl groups, such as trifluoromethyl, difluoromethyl, trifluoroethyl and the like.

The term “alkylalkoxy” refers to the groups -alkylene-O-alkyl, alkylene-O—substituted alkyl, substituted alkylene-O-alkyl, and substituted alkylene-O-substituted alkyl wherein alkyl, substituted alkyl, alkylene and substituted alkylene are as defined herein.

“Alkenyl” refers to straight chain or branched hydrocarbyl groups having from 2 to 6 carbon atoms and preferably 2 to 4 carbon atoms and having at least 1 and preferably from 1 to 2 sites of double bond unsaturation. This term includes, by way of example, bi-vinyl, allyl, and but-3-en-1-yl. Included within this term are the cis and trans isomers or mixtures of these isomers. The term “substituted alkenyl” refers to an alkenyl group as defined herein having from 1 to 5 substituents, or from 1 to 3 substituents. The term “alkenylene” refers to a divalent alkenyl group. Examples of alkenylene groups include alkylene and substituted alkylene groups having one or more alkene bonds in the hydrocarbon chain.

“Alkynyl” refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (—CH₂C≡CH). The term “substituted alkynyl” refers to an alkynyl group as defined herein having from 1 to 5 substituents, or from 1 to 3 substituents. The term “alkynylene” refers to divalent alkynyl groups (—C≡C—). Such groups may be included in a (C₂-C₁₂)alkylene or a substituted (C₁-C₁₂)alkylene, such as where an alkylene chain includes one or more alkyne bonds. Examples of such alkynylene groups include (—C≡C—), and (—CH₂C≡C—).

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—.

“Acylamino” refers to the groups comprising an amide moiety, namely, —NR²⁰C(O)alkyl, —NR²⁰C(O)substituted alkyl, N R²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyl” or the term “aminoacyl” refers to the amide group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the urea group, —NR²¹C(O)NR²²R²³, where R²¹, R²², and R²³ are independently selected from hydrogen, alkyl, aryl or cycloalkyl, or where two R groups are joined to form a heterocyclyl group.

The term “alkoxycarbonylamino” refers to the carbamate group, —NRC(O)OR, where each R is independently selected from hydrogen, alkyl, substituted alkyl, cycloalkyl, substituted cycloalkyl, aryl, substituted aryl, heteroaryl, substituted heteroaryl, heterocyclyl and substituted heterocyclyl wherein alkyl, substituted alkyl, cycloalkyl, substituted cycloalkyl, aryl, substituted aryl, heteroaryl, substituted heteroaryl, heterocyclyl and substituted heterocyclyl are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, substituted aryl-C(O)O—, heteroaryl-C(O)O—, substituted heteroaryl-C(O)O—, heterocyclyl-C(O)O—, and substituted heterocyclyl-C(O)O— wherein alkyl, substituted alkyl, cycloalkyl, substituted cycloalkyl, aryl, substituted aryl, heteroaryl, substituted heteroaryl, heterocyclyl and substituted heterocyclyl are as defined herein.

“Aminosulfonyl” refers to the group —SO₂NR^{c}R^{c}, wherein each R^{c} independently is hydrogen, R^{a}, or, alternatively, two R^{c} are taken together with the nitrogen atom to which they are bonded to form a 5 to 10-membered heterocyclylalkyl which may optionally include 1, 2 or 3 additional heteroatoms selected from O, N and S, and which may optionally be substituted.

“Sulfonylamino” refers to the group —NR²¹SO₂R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

“Aryloxy” refers to the group —O-aryl, wherein aryl is as defined herein, including, by way of example, phenoxy, naphthoxy, and the like, including optionally substituted aryl groups as also defined herein.

“Amino” refers to the group —NH₂.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

The term “azido” refers to the group —N₃.

“Carboxyl,” “carboxy” or “carboxylate” refers to —CO₂H or salts thereof.

“Carboxyl ester” or “carboxy ester” or the terms “carboxyalkyl” or “carboxylalkyl” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” or “carbonate” refers to the groups —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O— substituted aryl, —O—C(O)O-cycloalkyl, —O—C(O)O-substituted cycloalkyl, —O—C(O)O— cycloalkenyl, —O—C(O)O-substituted cycloalkenyl, —O—C(O)O-heteroaryl, —O—C(O)O— substituted heteroaryl, —O—C(O)O-heterocyclic, and —O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” or “nitrile” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like. The term “substituted cycloalkyl” refers to cycloalkyl groups having from 1 to 5 substituents, or from 1 to 3 substituents.

“Cycloalkenyl” refers to non-aromatic cyclic alkyl groups of from 3 to 10 carbon atoms having single or multiple rings and having at least one double bond and preferably from 1 to 2 double bonds.

“Cycloalkynyl” refers to non-aromatic cycloalkyl groups of from 5 to 10 carbon atoms having single or multiple rings and having at least one triple bond.

“Cycloalkoxy” refers to —O-cycloalkyl.

“Cycloalkenyloxy” refers to —O-cycloalkenyl.

“Halo” or “halogen” refers to fluoro, chloro, bromo, and iodo.

“Hydroxy” or “hydroxyl” refers to the group —OH.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic. To satisfy valence requirements, any heteroatoms in such heteroaryl rings may or may not be bonded to H or a substituent group, e.g., an alkyl group or other substituent as described herein. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents.

The term “heteroaralkyl” refers to the groups -alkylene-heteroaryl where alkylene and heteroaryl are defined herein. This term includes, by way of example, pyridylmethyl, pyridylethyl, indolylmethyl, and the like.

“Heteroaryloxy” refers to —O-heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from nitrogen, sulfur, or oxygen, where, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties. To satisfy valence requirements, any heteroatoms in such heterocyclic rings may or may not be bonded to one or more H or one or more substituent group(s), e.g., an alkyl group or other substituent as described herein.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents.

“Heterocyclyloxy” refers to the group —O-heterocyclyl.

The term “heterocyclylthio” refers to the group heterocyclic-S—.

The term “heterocyclene” refers to the diradical group formed from a heterocycle, as defined herein.

The term “hydroxyamino” refers to the group —NHOH.

“Nitro” refers to the group —NO₂.

“Oxo” refers to the atom (═O).

“Sulfonyl” refers to the group —SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, and —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cycloalkyl, —OSO₂-cycloalkenyl, —OSO₂-substituted cylcoalkenyl, —OSO₂-aryl, —OSO₂-substituted aryl, —OSO₂-heteroaryl, —OSO₂-substituted heteroaryl, —OSO₂-heterocyclic, and —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Sulfate” or “sulfate ester” refers the group —O—SO₂—OH, —O—SO₂—O-alkyl, —O—SO₂—O-substituted alkyl, —O—SO₂—O-alkenyl, —O—SO₂—O-substituted alkenyl, —O—SO₂—O-cycloalkyl, —O—SO₂—O-substituted cycloalkyl, —O—SO₂—O-cycloalkenyl, —O—SO₂—O-substituted cylcoalkenyl, —O—SO₂—O-aryl, —O—SO₂—O-substituted aryl, —O—SO₂—O-heteroaryl, —O—SO₂—O-substituted heteroaryl, —O—SO₂—O-heterocyclic, and —O—SO₂—O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “aminocarbonyloxy” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

“Thiol” refers to the group —SH.

“Thioxo” or the term “thioketo” refers to the atom (═S).

“Alkylthio” or the term “thioalkoxy” refers to the group —S-alkyl, wherein alkyl is as defined herein. In certain embodiments, sulfur may be oxidized to —S(O)—. The sulfoxide may exist as one or more stereoisomers.

The term “substituted thioalkoxy” refers to the group —S-substituted alkyl.

The term “thioaryloxy” refers to the group aryl-S— wherein the aryl group is as defined herein including optionally substituted aryl groups also defined herein.

The term “thioheteroaryloxy” refers to the group heteroaryl-S— wherein the heteroaryl group is as defined herein including optionally substituted aryl groups as also defined herein.

The term “thioheterocyclooxy” refers to the group heterocyclyl-S— wherein the heterocyclyl group is as defined herein including optionally substituted heterocyclyl groups as also defined herein.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR^{f}, ═N—OR^{f}, ═N₂ or ═S) on saturated carbon atoms in the specified group or radical are, unless otherwise specified, —R^{a}, R^{b}, and R^{a} substituted with one or more R^{a}, R^{b}, or both, where R^{a} is independently selected from hydrogen, C₁₋₈ alkyl, C₂₋₆ alkenyl, C₂₋₆ alkynyl, C₁₋₆ haloalkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl; R^{b} is independently selected from the group consisting of ═O, —OR^{a}, halogen, C₁₋₃haloalkyloxy, —OCF₃, ═S, —SR^{d}, ═NR^{d}, ═NOR^{d}, —NR^{c}R^{c}, —SF₅, halogen, —CF₃, —CN, —NO₂, —S(O)R^{d}, —S(O)₂R^{d}, —S(O)₂OR^{d}, —N(H)S(O)₂R^{d}, —S(O)NR^{c}R^{c}, —S(O)₂NR^{c}R^{c}, —OS(O)R^{d}, —OS(O)₂R^{d},

—OS(O)₂OR^{d}, —OS(O)₂NR^{c}R^{c}, —C(O)R^{d}, —C(O)OR^{d}, —C(O)NR^{c}R^{c},
—C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NH C(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —(CHR^{a}CHR^{a}—O)ₙ—R^{a}, —[NHC(NH)]ₙNR^{c}R^{c} and —[NR^{a}C(NR^{a})]ₙNR^{c}R^{c}; each R^{c} independently is hydrogen, R^{a}, or, alternatively, two R^{c} are taken together with the nitrogen atom to which they are bonded to form a 5 to 10-membered heterocyclylalkyl which may optionally include 1, 2 or 3 additional heteroatoms selected from O, N and S, and may optionally be substituted with one or more of the same or different R^{e} groups; each R^{d} is independently hydrogen or C₁₋₆ alkyl; R^{e} is independently selected from R^{d}, ═O, —OR^{a}, halogen, —C(O)R^{d}, and —S(O)₂R^{d}; each R^{f} is independently R^{a}, —C(O)R^{d}, or —S(O)₂R^{d}; and each n is independently an integer from 0 to 3.

In addition to the disclosure herein, substituent groups for hydrogens on unsaturated carbon atoms in “substituted” alkene, alkyne, aryl, heteroaryl, heteroalkyl and cycloheteroalkyl groups are, unless otherwise specified, —R^{a}, R^{b}, and R^{a} substituted with one or more R^{a}, R^{b}, or both.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment.

For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt. Salts of the present compounds also include those wherein the compound is deprotonated by an inorganic or organic base to form an anion, with the conjugate acid of the inorganic or organic base as the cationic component of the salt.

As will be recognized by those of skill in the art exemplary compounds may be present in the form of a salt. As such, reference to the present compounds, such compounds of formulas (I), (Ia), (Ib), (Ib1), (Ic), (Ic1), (Id), (Ie) and (If), includes reference to the salt forms of such compounds. Similarly, reference to the compounds of formulas (I), (Ia), (Ib), (Ib1), (Ic), (Ic1), (Id), (Ie) and (If), also includes reference to other non-covalently modified forms of the compounds, such as solvates of such compounds, for example hydrates.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

“Pharmaceutically effective amount” and “therapeutically effective amount” refer to an amount of a compound sufficient to treat a specified disorder or disease or one or more of its symptoms and/or to prevent the occurrence of the disease or disorder. In reference to tumorigenic proliferative disorders, a pharmaceutically or therapeutically effective amount comprises an amount sufficient to, among other things, cause the tumor to shrink or decrease the growth rate of the tumor.

“Patient” refers to human and non-human subjects, especially mammalian subjects.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the compositions and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Certain Embodiments

The present disclosure contemplates, among other things, the following numbered embodiments:
1. A method for inhibiting HIPK4, comprising contacting the HIPK4 with a compound of the formula:

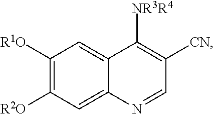

wherein
   R¹ is independently selected from hydrogen, —X-L^{A}-W, and C₁₋₆ alkyl optionally substituted with one or more R^{a}, R^{b}, or both;
   R² is independently selected from hydrogen, —X-L^{B}-W, and C₁₋₆ alkyl optionally substituted with one or more of the same or different R^{a}, R^{b}, or both;
   X is, for each occurrence, independently selected from a covalent bond,
   —C(O)—, —C(O)O—, —C(O)N(R^{d})—, and —S(O)₂—;
   L^{A} is a linker;
   L^{B} is a linker;
   W is a E3 ubiquitin ligase binding moiety;
   R³ and R⁴ are independently selected from hydrogen, C₁₋₆ alkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl, each optionally substituted with one or more of the same or different R^{a}, R^{b}, or both, or R³ and R⁴ are taken together with the nitrogen atom to which they are bonded to form a 5 to 8-membered cycloheteroalkyl or heteroaryl which may optionally include one or more of the same or different additional heteroatoms and which may optionally be substituted with one or more of the same or different which may optionally be substituted with one or more of the same or different R^{e} groups;
   each R^{a} is independently selected from hydrogen, C₁₋₆ alkyl, C₂₋₆ alkenyl, C₂₋₆ alkynyl, C₁₋₆ haloalkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl;
   R^{b} is independently selected from the group consisting of ═O, —OR^{a}, halogen, C₁₋₃haloalkyloxy, —OCF₃, ═S, —SR^{d}, ═NR^{d}, ═NOR^{d}, —NR^{c}R^{c}, —SF₅, halogen, —CF₃, —CN, —NO₂, —S(O)R^{d}, —S(O)₂R^{d}, —S(O)₂OR^{d},
   —N(H)S(O)₂R^{d}, —S(O)NR^{c}R^{c}, —S(O)₂NR^{c}R^{c}, —OS(O)R^{d}, —OS(O)₂R^{d},
   —OS(O)₂OR^{d}, —OS(O)₂NR^{c}R^{c}, —C(O)R^{d}, —C(O)OR^{d}, —C(O)NR^{c}R^{c},
   —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d},
   —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};
      each R^{c} independently is hydrogen, R^{a}, or, alternatively, two R^{c} are taken together with the nitrogen atom to which they are bonded to form a 5 to 10-membered heterocyclylalkyl which may optionally include 1, 2 or 3 additional heteroatoms selected from O, N and S, and may optionally be substituted with one or more of the same or different R^{e} groups;
   each R^{d} is independently hydrogen or C₁₋₆ alkyl;
   each R^{e} is independently R^{d}, ═O, —OR^{a}, halogen, —C(O)R^{d}, and —S(O)₂R^{d}; and
   each n is independently an integer from 0 to 3.
2. The method of embodiment 1, wherein R¹ is C₁₋₆ alkyl optionally substituted with one or more R^{a}, R^{b}, or both.
3. The method of embodiment 1 or 2, wherein R² is C₁₋₆ alkyl optionally substituted with one or more of the same or different R^{a}, R^{b}, or both.
4. The method of embodiment 1, wherein R¹ is -L^{A}-W.
5. The method of embodiment 4, wherein L^{A} comprises:

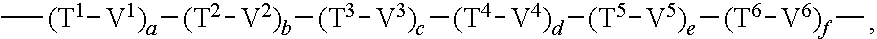

wherein
a, b, c, d, e and f are each independently 0 or 1;
T¹, T², T³, T⁴, T⁵ and T⁶ are each independently selected from a covalent bond, (C₁-C₁₂)alkylene, substituted (C₁-C₁₂)alkylene, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(C(R^{a})(OH))ₘ—, 4-amino-piperidine (4AP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n in the group (PEG)ₙ is an integer from 1 to 30, each p is an integer from 1 to 20, and each m in the group —(C(R^{a})(OH))ₘ— is an integer from 1 to 12;
V¹, V², V³, V⁴, V⁵ and V⁶ are each independently selected from the group consisting of a covalent bond, —C(O)—, —(CH₂)_{q}—C(O)—, —C(O)—(CH₂)_{q}—, —NR¹⁵—,
—NR¹⁵(CH₂)_{q}—, —NR¹⁵(C₆H₄)—, —(CH₂)_{q}—C(O)NR¹⁵—, —C(O)N(R¹⁵)—, —C(O)N(R¹⁵)—(CH₂)_{q}—, —NR¹⁵C(O)—, —(CH₂)_{q}—NR¹⁵C(O)—, —C(O)O—, —(CH₂)_{q}—C(O)O—, —OC(O)—, —OC(O)—(CH₂)_{q}—, —O—, —S—, —S(O)—, —SO₂—, —SO₂N(R¹⁵)—, —(CH₂)_{q}—SO₂N(R¹⁵)—, —NR¹⁵SO₂— and —P(O)OH—, wherein each q is an integer from 1 to 6; and
each R¹⁵ is independently selected from R^{a} and —C(O)R^{a}.
6. The method of embodiment 1, wherein R² is -L^{B}-W.
7. The method of embodiment 6, wherein -L^{B}- comprises:

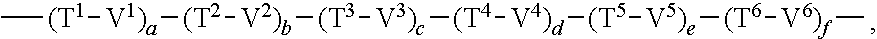

wherein
a, b, c, d, e and f are each independently 0 or 1;
T¹, T², T³, T⁴, T⁵ and T⁶ are each independently selected from a covalent bond, (C₁-C₁₂)alkylene, substituted (C₁-C₁₂)alkylene, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(C(R^{a})(OH))ₘ—, 4-amino-piperidine (4AP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each m is an integer from 1 to 12;
V¹, V², V³, V⁴, V⁵ and V⁶ are each independently selected from the group consisting of a covalent bond, —C(O)—, —(CH₂)_{q}—C(O)—, —C(O)—(CH₂)_{q}—, —NR¹⁵—,
—NR¹⁵(CH₂)_{q}—, —NR¹⁵(C₆H₄)—, —(CH₂)_{q}—C(O)NR¹⁵—, —C(O)N(R¹⁵)—, —C(O)N(R¹⁵)—(CH₂)_{q}—, —NR¹⁵C(O)—, —(CH₂)_{q}—NR¹⁵C(O)—, —C(O)O—, —(CH₂)_{q}—C(O)O—, —OC(O)—, —OC(O)—(CH₂)_{q}—, —O—, —S—, —S(O)—, —SO₂—, —SO₂N(R¹⁵)—, —(CH₂)_{q}—SO₂N(R¹⁵)—, —NR¹⁵SO₂— and —P(O)OH—, wherein each q is an integer from 1 to 6; and
each R¹⁵ is independently selected from R^{a} and —C(O)R^{a}.
8. The method of any one of embodiments 4-7, wherein W is a ligand for CRBN, VHL, IAP, MDM2, FEM1B, RNF114, or a combination thereof.
9. The method of embodiment 8, wherein W is a CRBN ligand.
10. The method of embodiment 9, wherein the CRBN ligand has a structure selected from:

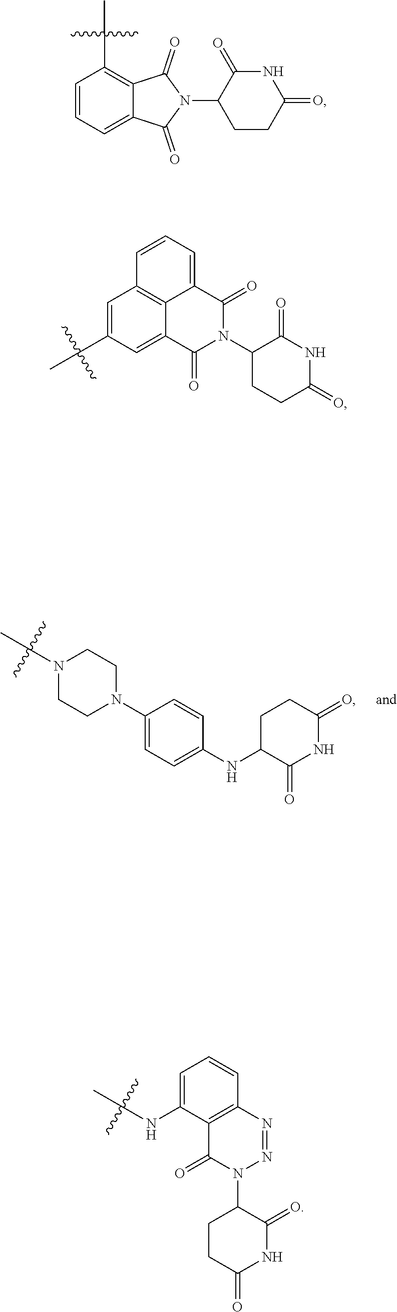 [SMILES: *c1cccc2c1C(=O)N(C1CCC(=O)NC1=O)C2=O ; *c1cc2c3c(cccc3c1)C(=O)N(C1CCC(=O)NC1=O)C2=O ; *N1CCN(c2ccc(NC3CCC(=O)NC3=O)cc2)CC1 ; "and" ; *Nc1cccc2nnn(C3CCC(=O)NC3=O)c(=O)c12]

11. The method of embodiment 8, wherein W is a VHL ligand.
12. The method of embodiment 11, wherein the VHL ligand has a structure

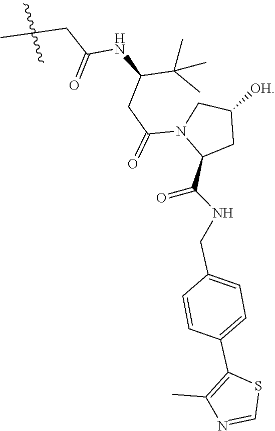 [SMILES: *CC(=O)N[C@H](CC(=O)N1C[C@H](O)C[C@H]1C(=O)NCc1ccc(-c2scnc2C)cc1)C(C)(C)C]

13. The method of embodiment 8, wherein W is a FEM1B ligand.
14. The method of embodiment 13 wherein the FEM1B ligand has a structure:

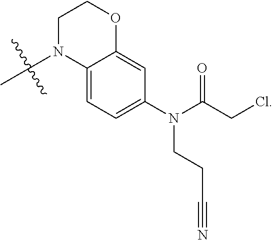 [SMILES: *N1CCOc2cc(N(CCC#N)C(=O)CCl)ccc21]

15. The method of embodiment 8, wherein W is an RNF114 ligand.
16. The method of embodiment 15, wherein the RNF114 ligand has a structure:

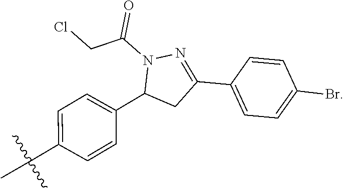 [SMILES: *c1ccc(C2CC(c3ccc(Br)cc3)=NN2C(=O)CCl)cc1]

17. The method of any one of embodiments 1-3, wherein the compound has the formula

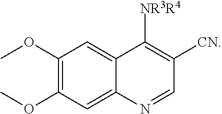

18. The method of any one of embodiments 1-17, wherein one of R³ and R⁴ is hydrogen.
19. The method of any one of embodiments 1-18, wherein one of R³ and R⁴ is selected from C₁₋₆ alkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl, each optionally substituted with one or more of the same or different R^{a}, R^{b}.
20. The method of any one of embodiments 1-19, wherein contacting the HIPK4 induces HIPK4 degradation.
21. The method of embodiment 1, wherein the compound has the formula

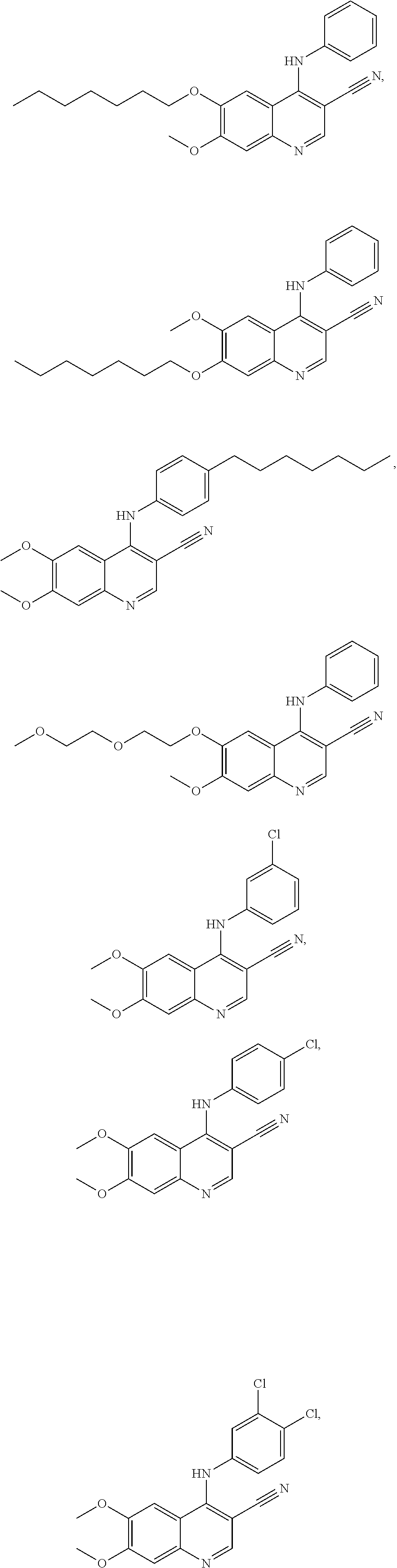

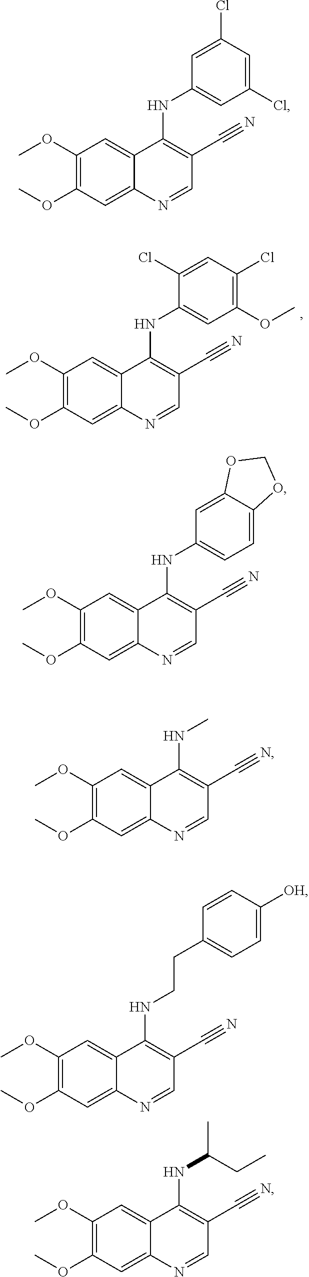

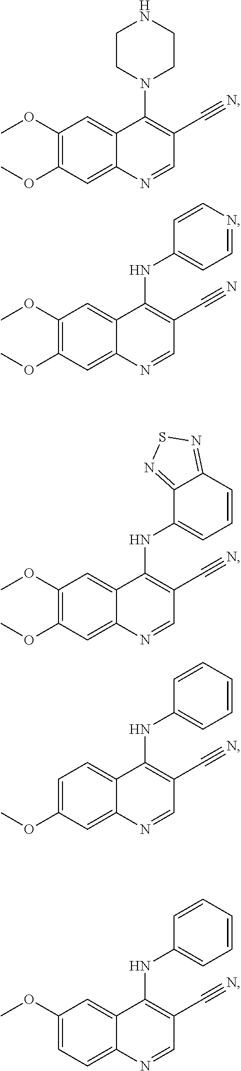

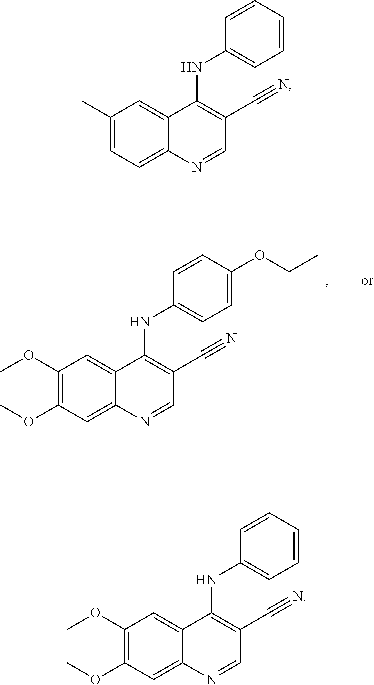

22. A method for inhibiting spermiogenesis, comprising administering an effective amount of a compound according to the formula:

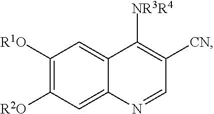

wherein
   R¹ is independently selected from hydrogen, —X-L^{A}-W, and C₁₋₆ alkyl optionally substituted with one or more R^{a}, R^{b}, or both;
   R² is independently selected from hydrogen, —X-L^{B}-W, and C₁₋₆ alkyl optionally substituted with one or more of the same or different R^{a}, R^{b}, or both;
   X is, for each occurrence, independently selected from a covalent bond,
   —C(O)—, —C(O)O—, —C(O)N(R^{d})—, and —S(O)₂—;
   L^{A} is a linker;
   L^{B} is a linker;
   W is a E3 ubiquitin ligase binding moiety;
   R³ and R⁴ are independently selected from hydrogen, C₁₋₆ alkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl, each optionally substituted with one or more of the same or different R^{a}, R^{b}, or both, or R³ and R⁴ are taken together with the nitrogen atom to which they are bonded to form a 5 to 8-membered cycloheteroalkyl or heteroaryl which may optionally include one or more of the same or different additional heteroatoms and which may optionally be substituted with one or more of the same or different which may optionally be substituted with one or more of the same or different R^{e} groups;
   each R^{a} is independently selected from hydrogen, C₁₋₆ alkyl, C₂₋₆ alkenyl, C₂₋₆ alkynyl, C₁₋₆ haloalkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl;
   R^{b} is independently selected from the group consisting of ═O, —OR^{a}, halogen, C₁₋₃haloalkyloxy, —OCF₃, ═S, —SR^{d}, ═NR^{d}, ═NOR^{d}, —NR^{c}R^{c}, —SF₅, halogen, —CF₃, —CN, —NO₂, —S(O)R^{d}, —S(O)₂R^{d}, —S(O)₂OR^{d},
   —N(H)S(O)₂R^{d}, —S(O)NR^{c}R^{c}, —S(O)₂NR^{c}R^{c}, —OS(O)R^{d}, —OS(O)₂R^{d},
   —OS(O)₂OR^{d}, —OS(O)₂NR^{c}R^{c}, —C(O)R^{d}, —C(O)OR^{d}, —C(O)NR^{c}R^{c},
   —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d},
   —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};
      each R^{c} independently is hydrogen, R^{a}, or, alternatively, two R^{c} are taken together with the nitrogen atom to which they are bonded to form a 5 to 10-membered heterocyclylalkyl which may optionally include 1, 2 or 3 additional heteroatoms selected from O, N and S, and may optionally be substituted with one or more of the same or different R^{e} groups;
   each R^{d} is independently hydrogen or C₁₋₆ alkyl;
   each R^{e} is independently R^{d}, ═O, —OR^{a}, halogen, —C(O)R^{d}, and —S(O)₂R^{d}; and
   each n is independently an integer from 0 to 3.
23. A compound of the formula:

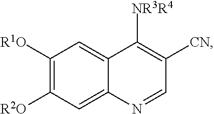

wherein
   R¹ is independently selected from hydrogen, —X-L^{A}-W, and C₁₋₆ alkyl optionally substituted with one or more R^{a}, R^{b}, or both;
   R² is independently selected from hydrogen, —X-L^{B}-W, and C₁₋₆ alkyl optionally substituted with one or more of the same or different R^{a}, R^{b}, or both; provided that when R¹ is hydrogen or C₁₋₆ alkyl optionally substituted with one or more R^{a}, R^{b}, or both, R² is —X-L^{B}-W, and when R² is hydrogen or C₁₋₆ alkyl optionally substituted with one or more R^{a}, R^{b}, or both, R¹ is —X-L^{A}-W;
   X is, for each occurrence, independently selected from a covalent bond,
   —C(O)—, —C(O)O—, —C(O)N(R^{d})—, and —S(O)₂—;
   L^{A} is a linker;
   L^{B} is a linker;
   W is a E3 ubiquitin ligase binding moiety;
   R³ and R⁴ are independently selected from hydrogen, C₁₋₆ alkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl, each optionally substituted with one or more of the same or different R^{a}, R^{b}, or both, or R³ and R⁴ are taken together with the nitrogen atom to which they are bonded to form a 5 to 8-membered cycloheteroalkyl or heteroaryl which may optionally include one or more of the same or different additional heteroatoms and which may optionally be substituted with one or more of the same or different which may optionally be substituted with one or more of the same or different R^{e} groups;
   each R^{a} is independently selected from hydrogen, C₁₋₆ alkyl, C₂₋₆ alkenyl, C₂₋₆ alkynyl, C₁₋₆ haloalkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl;
   R^{b} is independently selected from the group consisting of ═O, —OR^{a}, halogen, C₁₋₃haloalkyloxy, —OCF₃, ═S, —SR^{d}, ═NR^{d}, ═NOR^{d}, —NR^{c}R^{c}, —SF₅, halogen, —CF₃, —CN, —NO₂, —S(O)R^{d}, —S(O)₂R^{d}, —S(O)₂OR^{d},
   —N(H)S(O)₂R^{d}, —S(O)NR^{c}R^{c}, —S(O)₂NR^{c}R^{c}, —OS(O)R^{d}, —OS(O)₂R^{d},
   —OS(O)₂OR^{d}, —OS(O)₂NR^{c}R^{c}, —C(O)R^{d}, —C(O)OR^{d}, —C(O)NR^{c}R^{c},
   —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d},
   —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};
      each R^{c} independently is hydrogen, R^{a}, or, alternatively, two R^{c} are taken together with the nitrogen atom to which they are bonded to form a 5 to 10-membered heterocyclylalkyl which may optionally include 1, 2 or 3 additional heteroatoms selected from O, N and S, and may optionally be substituted with one or more of the same or different R^{e} groups;
   each R^{d} is independently hydrogen or C₁₋₆ alkyl;
   each R^{e} is independently R^{d}, ═O, —OR^{a}, halogen, —C(O)R^{d}, and —S(O)₂R^{d}; and
   each n is independently an integer from 0 to 3.
24. The compound of embodiment 23, wherein R¹ is —X-L^{A}-W.
25. The compound of embodiment 24, wherein L^{A} comprises:

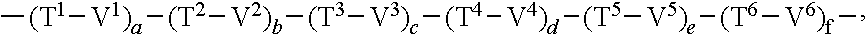

wherein
a, b, c, d, e and f are each independently 0 or 1;
T¹, T², T³, T⁴, T⁵ and T⁶ are each independently selected from a covalent bond, (C₁-C₁₂)alkylene, substituted (C₁-C₁₂)alkylene, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(C(R^{a})(OH))ₘ—, 4-amino-piperidine (4AP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n in the group (PEG)ₙ is an integer from 1 to 30, each p is an integer from 1 to 20, and each m in the group —(C(R^{a})(OH))ₘ— is an integer from 1 to 12;
V¹, V², V³, V⁴, V⁵ and V⁶ are each independently selected from the group consisting of a covalent bond, —C(O)—, —(CH₂)_{q}—C(O)—, —C(O)—(CH₂)_{q}—, —NR¹⁵—,
—NR¹⁵(CH₂)_{q}—, —NR¹⁵(C₆H₄)—, —(CH₂)_{q}—C(O)NR¹⁵—, —C(O)N(R¹⁵)—, —C(O)N(R¹⁵)—(CH₂)_{q}—, —NR¹⁵C(O)—, —(CH₂)_{q}—NR¹⁵C(O)—, —C(O)O—, —(CH₂)_{q}—C(O)O—, —OC(O)—, —OC(O)—(CH₂)_{q}—, —O—, —S—, —S(O)—, —SO₂—, —SO₂N(R¹⁵)—, —(CH₂)_{q}—SO₂N(R¹⁵)—, —NR¹⁵SO₂— and —P(O)OH—, wherein each q is an integer from 1 to 6; and
each R¹⁵ is independently selected from R^{a} and —C(O)R^{a}.
26. The compound of embodiment 23, wherein R²—X-L^{B}-W.
27. The compound of embodiment 26, wherein -L^{B}- comprises:

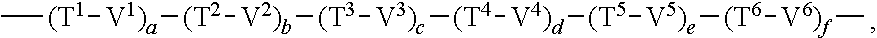

wherein
a, b, c, d, e and f are each independently 0 or 1;
T¹, T², T³, T⁴, T⁵ and T⁶ are each independently selected from a covalent bond, (C₁-C₁₂)alkylene, substituted (C₁-C₁₂)alkylene, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(C(R^{a})(OH))ₘ—, 4-amino-piperidine (4AP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each m is an integer from 1 to 12;
V¹, V², V³, V⁴, V⁵ and V⁶ are each independently selected from the group consisting of a covalent bond, —C(O)—, —(CH₂)_{q}—C(O)—, —C(O)—(CH₂)_{q}—, —NR¹⁵—,
—NR¹⁵(CH₂)_{q}—, —NR¹⁵(C₆H₄)—, —(CH₂)_{q}—C(O)NR¹⁵—, —C(O)N(R¹⁵)—, —C(O)N(R¹⁵)—(CH₂)_{q}—, —NR¹⁵C(O)—, —(CH₂)_{q}—NR¹⁵C(O)—, —C(O)O—, —(CH₂)_{q}—C(O)O—, —OC(O)—, —OC(O)—(CH₂)_{q}—, —O—, —S—, —S(O)—, —SO₂—, —SO₂N(R¹⁵)—, —(CH₂)_{q}—SO₂N(R¹⁵)—, —NR¹⁵SO₂— and —P(O)OH—, wherein each q is an integer from 1 to 6; and
each R¹⁵ is independently selected from R^{a} and —C(O)R^{a}.
28. The compound of any one of embodiments 23-27, wherein the linker L^{A}, L^{B}, or both
   have the formula

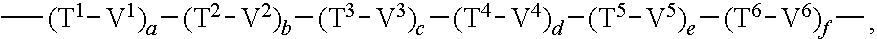

wherein
T¹ is selected from (PEG)ₙ, wherein n is 1 or 2;
V¹ is —NR¹⁵—;
R¹⁵ is hydrogen; and
b, c, d, e and f are each 0.
29. The compound of any one of embodiments 23-27, wherein the linker L^{A}, L^{B}, or both
   have the formula

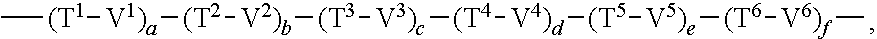

wherein
a and b are each 1, and c, d, e and fare each 0;
T¹ is selected from (PEG)ₙ, wherein n is 1 or 2;
V¹ is —C(O)N(R¹⁵)—;
R¹⁵ is hydrogen;
T² is (C₁-C₁₂)alkyl; and
V² is —O—.
30. The compound of any one of embodiments 23-29, wherein W is a ligand for CRBN, VHL, IAP, MDM2, FEM1B, RNF114, or a combination thereof.
31. The compound of embodiment 30, wherein W is a CRBN ligand.
32. The compound of embodiment 31, wherein the CRBN ligand has a structure selected from:

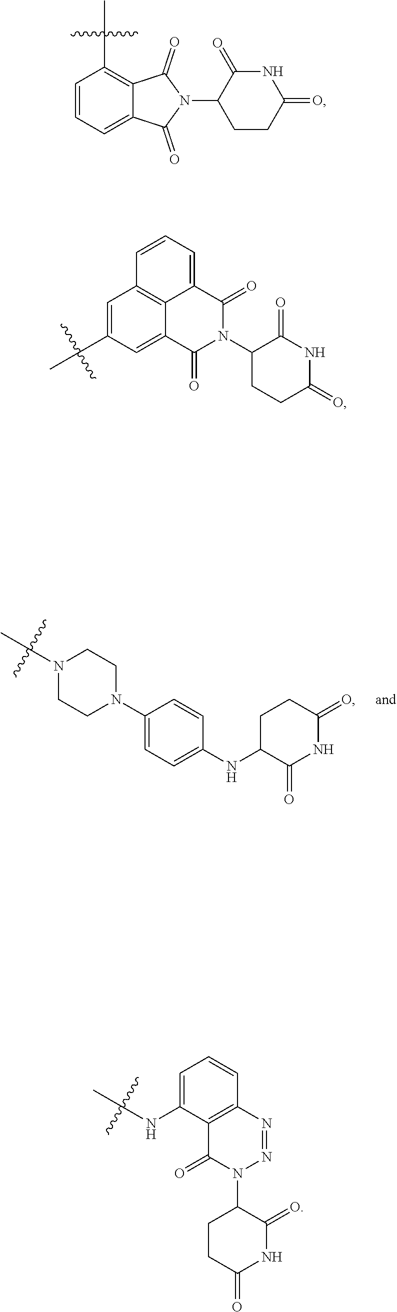 [SMILES: *c1cccc2c1C(=O)N(C1CCC(=O)NC1=O)C2=O ; *c1cc2c3c(cccc3c1)C(=O)N(C1CCC(=O)NC1=O)C2=O ; *N1CCN(c2ccc(NC3CCC(=O)NC3=O)cc2)CC1 ; "and" ; *Nc1cccc2nnn(C3CCC(=O)NC3=O)c(=O)c12]

33. The compound of embodiment 30, wherein W is a VHL ligand.
34. The compound embodiment 33, wherein the VHL ligand has a structure

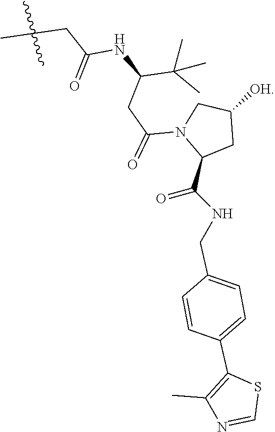 [SMILES: *CC(=O)N[C@H](CC(=O)N1C[C@H](O)C[C@H]1C(=O)NCc1ccc(-c2scnc2C)cc1)C(C)(C)C]

35. The compound of embodiment 30, wherein W is a FEM1B ligand.
36. The compound of embodiment 35, wherein the FEM1B ligand has a structure:

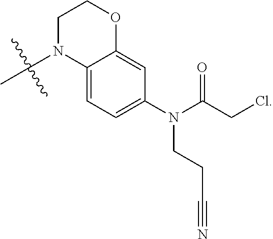 [SMILES: *N1CCOc2cc(N(CCC#N)C(=O)CCl)ccc21]

37. The compound of embodiment 30, wherein W is an RNF114 ligand.
38. The compound of embodiment 37, wherein the RNF114 ligand has a structure:

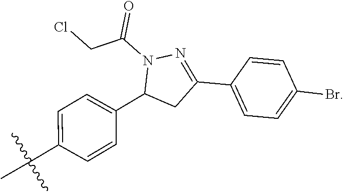 [SMILES: *c1ccc(C2CC(c3ccc(Br)cc3)=NN2C(=O)CCl)cc1]

39. A pharmaceutical composition comprising an effective amount of a compound of any one of embodiments 23-38.
40. The compound of any one of embodiments 23-38 in the form of a pharmaceutically acceptable salt.

### Further Embodiments

The present disclosure also contemplates, among other things, the following further numbered embodiments:
1. A method for inhibiting HIPK4, comprising contacting the HIPK4 with a compound of the formula:

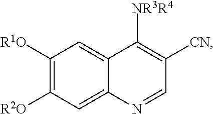

wherein
   R¹ is independently selected from hydrogen, —X-L^{A}-W, and C₁₋₆ alkyl optionally substituted with one or more R^{a}, R^{b}, or both;
   R² is independently selected from hydrogen, —X-L^{B}-W, and C₁₋₆ alkyl optionally substituted with one or more of the same or different R^{a}, R^{b}, or both;
   X is, for each occurrence, independently selected from a covalent bond,
   —C(O)—, —C(O)O—, —C(O)N(R^{d})—, and —S(O)₂—;
   L^{A} is a linker;
   L^{B} is a linker;
   W is a E3 ubiquitin ligase binding moiety;
   R³ and R⁴ are independently selected from hydrogen, C₁₋₆ alkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl, each optionally substituted with one or more of the same or different R^{a}, R^{b}, or both, or R³ and R⁴ are taken together with the nitrogen atom to which they are bonded to form a 5 to 8-membered cycloheteroalkyl or heteroaryl which may optionally include one or more of the same or different additional heteroatoms and which may optionally be substituted with one or more of the same or different which may optionally be substituted with one or more of the same or different R^{e} groups;
   each R^{a} is independently selected from hydrogen, C₁₋₈ alkyl, C₂₋₆ alkenyl, C₂₋₆ alkynyl, C₁₋₆ haloalkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl;
   R^{b} is independently selected from the group consisting of ═O, —OR^{a}, halogen, C₁₋₃haloalkyloxy, —OCF₃, ═S, —SR^{d}, ═NR^{d}, ═NOR^{d}, —NR^{c}R^{c}, —SF₅, halogen, —CF₃, —CN, —NO₂, —S(O)R^{d}, —S(O)₂R^{d}, —S(O)₂OR^{d},
   —N(H)S(O)₂R^{d}, —S(O)NR^{c}R^{c}, —S(O)₂NR^{c}R^{c}, —OS(O)R^{d}, —OS(O)₂R^{d},
   —OS(O)₂OR^{d}, —OS(O)₂NR^{c}R^{c}, —C(O)R^{d}, —C(O)OR^{d}, —C(O)NR^{c}R^{c},
   —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d},
   —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —(CHR^{a}CHR^{a}—O)ₙ—R^{a}, —[NHC(NH)]ₙNR^{c}R^{c} and —[NR^{a}C(NR^{a})]ₙNR^{c}R^{c};
      each R^{c} independently is hydrogen, R^{a}, or, alternatively, two R^{c} are taken together with the nitrogen atom to which they are bonded to form a 5 to 10-membered heterocyclylalkyl which may optionally include 1, 2 or 3 additional heteroatoms selected from O, N and S, and may optionally be substituted with one or more of the same or different R^{e} groups;
   each R^{d} is independently hydrogen or C₁₋₆ alkyl;
   each R^{e} is independently R^{d}, ═O, —OR^{a}, halogen, —C(O)R^{d}, and —S(O)₂R^{d}; and
   each n is independently an integer from 0 to 3.
2. The method of embodiment 1, wherein R¹ is C₁₋₆ alkyl optionally substituted with one or more R^{a}, R^{b}, or both.
3. The method of embodiment 1 or 2, wherein R² is C₁₋₆ alkyl optionally substituted with one or more of the same or different R^{a}, R^{b}, or both.
4. The method of embodiment 1, wherein R¹ is -L^{A}-W.
5. The method of embodiment 4, wherein L^{A} comprises:

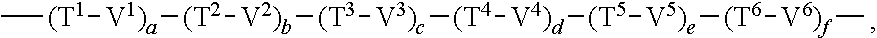

wherein
a, b, c, d, e and f are each independently 0 or 1;
T¹, T², T³, T⁴, T⁵ and T⁶ are each independently selected from a covalent bond, (C₁-C₁₂)alkylene, substituted (C₁-C₁₂)alkylene, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(C(R^{a})(OH))ₘ—, 4-amino-piperidine (4AP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n in the group (PEG)ₙ is an integer from 1 to 30, each p is an integer from 1 to 20, and each m in the group —(C(R^{a})(OH))ₘ— is an integer from 1 to 12;
V¹, V², V³, V⁴, V⁵ and V⁶ are each independently selected from the group consisting of a covalent bond, —C(O)—, —(CH₂)_{q}—C(O)—, —C(O)—(CH₂)_{q}—, —NR¹⁵—,
—NR¹⁵(CH₂)_{q}—, —NR¹⁵(C₆H₄)—, —(CH₂)_{q}—C(O)NR¹⁵—, —C(O)N(R¹⁵)—, —C(O)N(R¹⁵)—(CH₂)_{q}—, —NR¹⁵C(O)—, —(CH₂)_{q}—NR¹⁵C(O)—, —C(O)O—, —(CH₂)_{q}—C(O)O—, —OC(O)—, —OC(O)—(CH₂)_{q}—, —O—, —S—, —S(O)—, —SO₂—, —SO₂N(R¹⁵)—, —(CH₂)_{q}—SO₂N(R¹⁵)—, —NR¹⁵SO₂— and —P(O)OH—, wherein each q is an integer from 1 to 6; and
each R¹⁵ is independently selected from R^{a} and —C(O)R^{a}.
6. The method of embodiment 1, wherein R² is -L^{B}-W.
7. The method of embodiment 6, wherein -L^{B}- comprises:

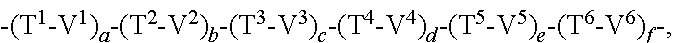

wherein
a, b, c, d, e and f are each independently 0 or 1;
T¹, T², T³, T⁴, T⁵ and T⁶ are each independently selected from a covalent bond, (C₁-C₁₂)alkylene, substituted (C₁-C₁₂)alkylene, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(C(R^{a})(OH))ₘ—, 4-amino-piperidine (4AP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each m is an integer from 1 to 12;
V¹, V², V³, V⁴, V⁵ and V⁶ are each independently selected from the group consisting of a covalent bond, —C(O)—, —(CH₂)_{q}—C(O)—, —C(O)—(CH₂)_{q}—, —NR¹⁵—,
—NR¹⁵(CH₂)_{q}—, —NR¹⁵(C₆H₄)—, —(CH₂)_{q}—C(O)NR¹⁵—, —C(O)N(R¹⁵)—, —C(O)N(R¹⁵)—(CH₂)_{q}—, —NR¹⁵C(O)—, —(CH₂)_{q}—NR¹⁵C(O)—, —C(O)O—, —(CH₂)_{q}—C(O)O—, —OC(O)—, —OC(O)—(CH₂)_{q}—, —O—, —S—, —S(O)—, —SO₂—, —SO₂N(R¹⁵)—, —(CH₂)_{q}—SO₂N(R¹⁵)—, —NR¹⁵SO₂— and —P(O)OH—, wherein each q is an integer from 1 to 6; and
each R¹⁵ is independently selected from R^{a} and —C(O)R^{a}.
8. The method of any one of embodiments 4-7, wherein W is a ligand for CRBN, VHL, IAP, MDM2, FEM1B, RNF114, or a combination thereof.
9. The method of embodiment 8, wherein W is a CRBN ligand.
10. The method of embodiment 9, wherein the CRBN ligand has a structure selected from:

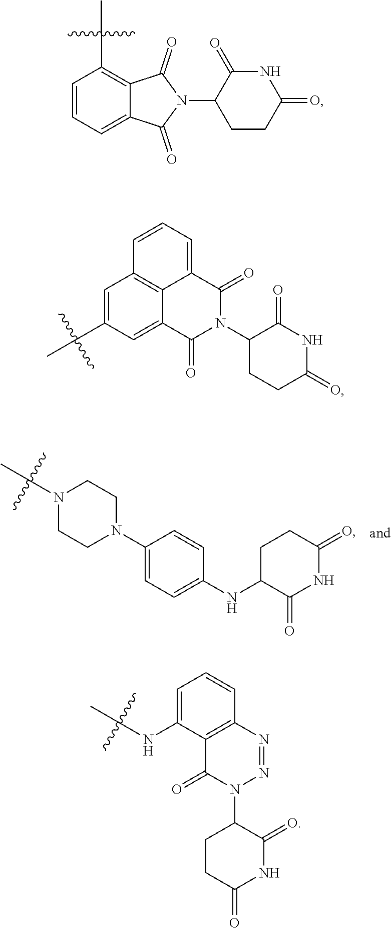 [SMILES: *c1cccc2c1C(=O)N(C1CCC(=O)NC1=O)C2=O ; *c1cc2c3c(cccc3c1)C(=O)N(C1CCC(=O)NC1=O)C2=O ; *N1CCN(c2ccc(NC3CCC(=O)NC3=O)cc2)CC1 ; *Nc1cccc2nnn(C3CCC(=O)NC3=O)c(=O)c12]

11. The method of embodiment 8, wherein W is a VHL ligand.
12. The method of embodiment 11, wherein the VHL ligand has a structure

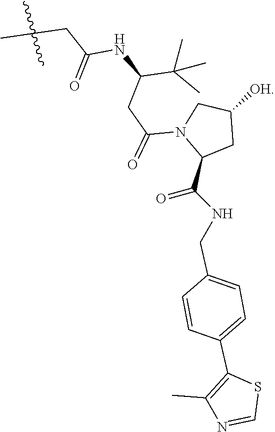 [SMILES: *CC(=O)N[C@H](CC(=O)N1C[C@H](O)C[C@H]1C(=O)NCc1ccc(-c2scnc2C)cc1)C(C)(C)C]

13. The method of embodiment 8, wherein W is a FEM1B ligand.
14. The method of embodiment 13 wherein the FEM1B ligand has a structure:

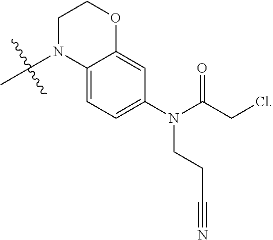 [SMILES: *N1CCOc2cc(N(CCC#N)C(=O)CCl)ccc21]

15. The method of embodiment 8, wherein W is an RNF114 ligand.
16. The method of embodiment 15, wherein the RNF114 ligand has a structure:

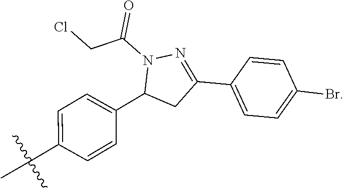 [SMILES: *c1ccc(C2CC(c3ccc(Br)cc3)=NN2C(=O)CCl)cc1]

17. The method of any one of embodiments 1-3, wherein the compound has the formula

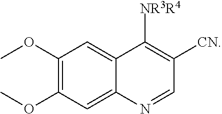

18. The method of any one of embodiments 1-17, wherein one of R³ and R⁴ is hydrogen.
19. The method of any one of embodiments 1-18, wherein one of R³ and R⁴ is selected from C₁₋₆ alkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl, each optionally substituted with one or more of the same or different R^{a}, R^{b}.
20. The method of any one of embodiments 1-19, wherein contacting the HIPK4 induces HIPK4 degradation.
21. The method of embodiment 1, wherein the compound has the formula

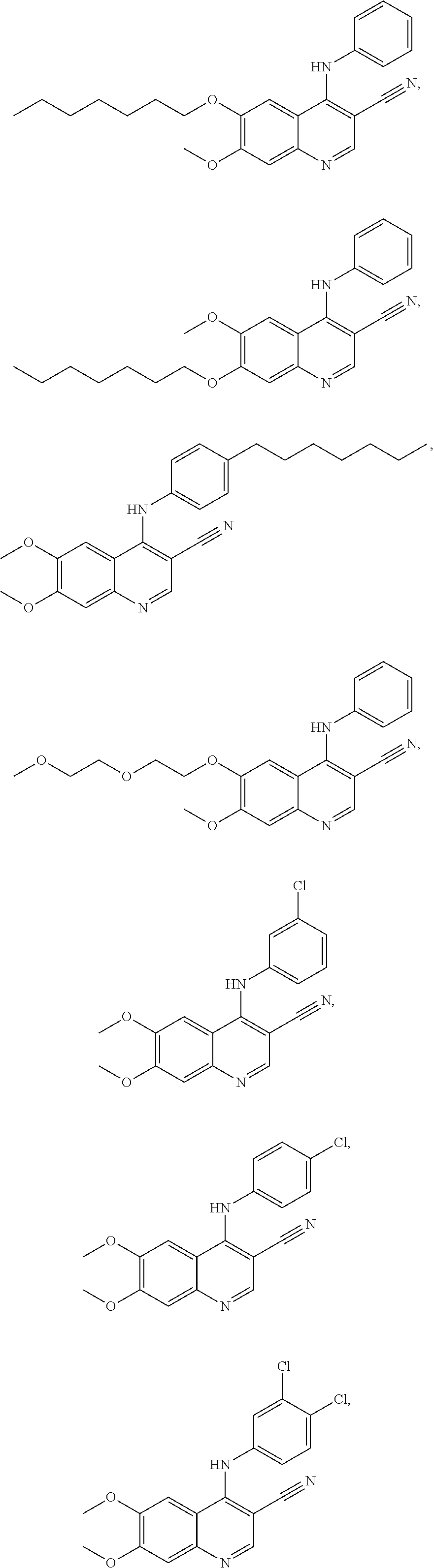

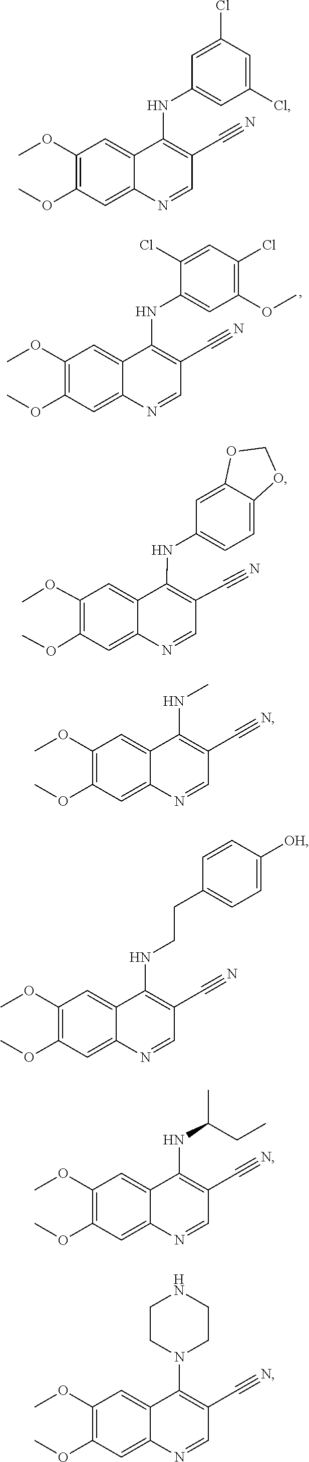

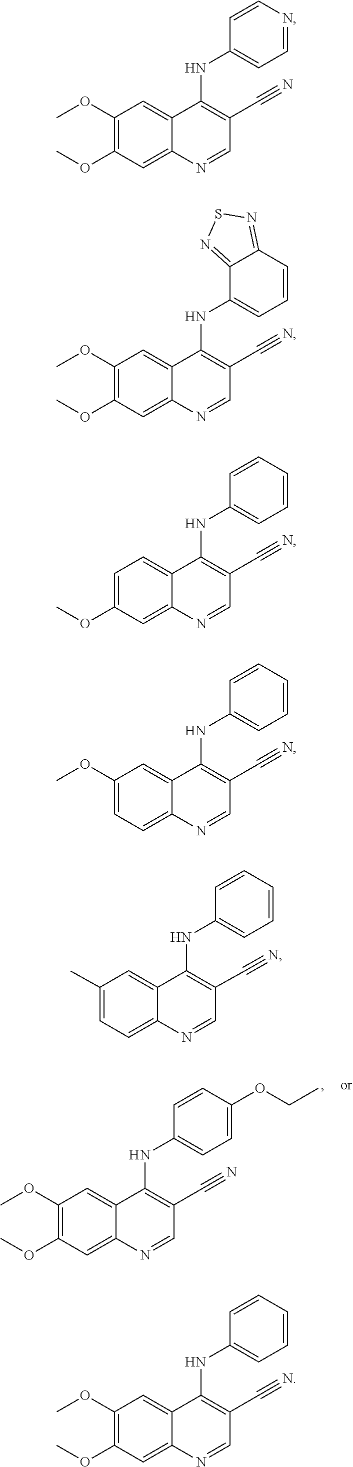

22. A method for inhibiting spermiogenesis, comprising administering an effective amount of a compound according to the formula:

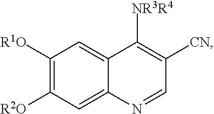

wherein
   R¹ is independently selected from hydrogen, —X-L^{A}-W, and C₁₋₆ alkyl optionally substituted with one or more R^{a}, R^{b}, or both;
   R² is independently selected from hydrogen, —X-L^{B}-W, and C₁₋₆ alkyl optionally substituted with one or more of the same or different R^{a}, R^{b}, or both;
   X is, for each occurrence, independently selected from a covalent bond,
   —C(O)—, —C(O)O—, —C(O)N(R^{d})—, and —S(O)₂—;
   L^{A} is a linker;
   L^{B} is a linker;
   W is a E3 ubiquitin ligase binding moiety;
   R³ and R⁴ are independently selected from hydrogen, C₁₋₆ alkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl, each optionally substituted with one or more of the same or different R^{a}, R^{b}, or both, or R³ and R⁴ are taken together with the nitrogen atom to which they are bonded to form a 5 to 8-membered cycloheteroalkyl or heteroaryl which may optionally include one or more of the same or different additional heteroatoms and which may optionally be substituted with one or more of the same or different which may optionally be substituted with one or more of the same or different R^{e} groups;
   each R^{a} is independently selected from hydrogen, C₁₋₈ alkyl, C₂₋₆ alkenyl, C₂₋₆ alkynyl, C₁₋₆ haloalkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl;
   R^{b} is independently selected from the group consisting of ═O, —OR^{a}, halogen, C₁₋₃haloalkyloxy, —OCF₃, ═S, —SR^{d}, ═NR^{d}, ═NOR^{d}, —NR^{c}R^{c}, —SF₅, halogen, —CF₃, —CN, —NO₂, —S(O)R^{d}, —S(O)₂R^{d}, —S(O)₂OR^{d},
   —N(H)S(O)₂R^{d}, —S(O)NR^{c}R^{c}, —S(O)₂NR^{c}R^{c}, —OS(O)R^{d}, —OS(O)₂R^{d},
   —OS(O)₂OR^{d}, —OS(O)₂NR^{c}R^{c}, —C(O)R^{d}, —C(O)OR^{d}, —C(O)NR^{c}R^{c},
   —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d},
   —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —(CHR^{a}CH R^{a}—O)ₙ—R^{a}, —[NHC(NH)]ₙNR^{c}R^{c} and —[NR^{a}C(NR^{a})]ₙNR^{c}R^{c};
      each R^{c} independently is hydrogen, R^{a}, or, alternatively, two R^{c} are taken together with the nitrogen atom to which they are bonded to form a 5 to 10-membered heterocyclylalkyl which may optionally include 1, 2 or 3 additional heteroatoms selected from O, N and S, and may optionally be substituted with one or more of the same or different R^{e} groups;
   each R^{d} is independently hydrogen or C₁₋₆ alkyl;
   each R^{e} is independently R^{d}, ═O, —OR^{a}, halogen, —C(O)R^{d}, and —S(O)₂R^{d}; and
   each n is independently an integer from 0 to 3.
23. A compound of the formula:

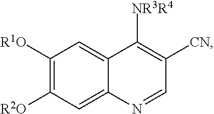

wherein
   R¹ is independently selected from hydrogen, —X-L^{A}-W, and C₁₋₆ alkyl optionally substituted with one or more R^{a}, R^{b}, or both;
   R² is independently selected from hydrogen, —X-L^{B}-W, and C₁₋₆ alkyl optionally substituted with one or more of the same or different R^{a}, R^{b}, or both; provided that when R¹ is hydrogen or C₁₋₆ alkyl optionally substituted with one or more R^{a}, R^{b}, or both, R² is —X-L^{B}-W, and when R² is hydrogen or C₁₋₆ alkyl optionally substituted with one or more R^{a}, R^{b}, or both, R¹ is —X-L^{A}-W;
   X is, for each occurrence, independently selected from a covalent bond,
   —C(O)—, —C(O)O—, —C(O)N(R^{d})—, and —S(O)₂—;
   L^{A} is a linker;
   L^{B} is a linker;
   W is a E3 ubiquitin ligase binding moiety;
   R³ and R⁴ are independently selected from hydrogen, C₁₋₆ alkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl, each optionally substituted with one or more of the same or different R^{a}, R^{b}, or both, or R³ and R⁴ are taken together with the nitrogen atom to which they are bonded to form a 5 to 8-membered cycloheteroalkyl or heteroaryl which may optionally include one or more of the same or different additional heteroatoms and which may optionally be substituted with one or more of the same or different which may optionally be substituted with one or more of the same or different R^{e} groups;
   each R^{a} is independently selected from hydrogen, C₁₋₈ alkyl, C₂₋₆ alkenyl, C₂₋₆ alkynyl, C₁₋₆ haloalkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl;
   R^{b} is independently selected from the group consisting of ═O, —OR^{a}, halogen, C₁₋₃haloalkyloxy, —OCF₃, ═S, —SR^{d}, ═NR^{d}, ═NOR^{d}, —NR^{c}R^{c}, —SF₅, halogen, —CF₃, —CN, —NO₂, —S(O)R^{d}, —S(O)₂R^{d}, —S(O)₂OR^{d},
   —N(H)S(O)₂R^{d}, —S(O)NR^{c}R^{c}, —S(O)₂NR^{c}R^{c}, —OS(O)R^{d}, —OS(O)₂R^{d},
   —OS(O)₂OR^{d}, —OS(O)₂NR^{c}R^{c}, —C(O)R^{d}, —C(O)OR^{d}, —C(O)NR^{c}R^{c},
   —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d},
   —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —(CHR^{a}CH R^{a}—O)ₙ—R^{a}, —[NHC(NH)]ₙNR^{c}R^{c} and —[NR^{a}C(NR^{a})]ₙNR^{c}R^{c};
      each R^{c} independently is hydrogen, R^{a}, or, alternatively, two R^{c} are taken together with the nitrogen atom to which they are bonded to form a 5 to 10-membered heterocyclylalkyl which may optionally include 1, 2 or 3 additional heteroatoms selected from O, N and S, and may optionally be substituted with one or more of the same or different R^{e} groups;
   each R^{d} is independently hydrogen or C₁₋₆ alkyl;
   each R^{e} is independently R^{d}, ═O, —OR^{a}, halogen, —C(O)R^{d}, and —S(O)₂R^{d}; and
   each n is independently an integer from 0 to 3.
24. The compound of embodiment 23, wherein R¹ is —X-L^{A}-W.
25. The compound of embodiment 24, wherein L^{A} comprises:

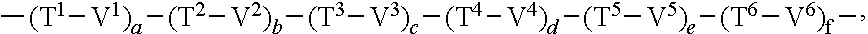

wherein
a, b, c, d, e and f are each independently 0 or 1;
T¹, T², T³, T⁴, T⁵ and T⁶ are each independently selected from a covalent bond, (C₁-C₁₂)alkylene, substituted (C₁-C₁₂)alkylene, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(C(R^{a})(OH))ₘ—, 4-amino-piperidine (4AP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n in the group (PEG)ₙ is an integer from 1 to 30, each p is an integer from 1 to 20, and each m in the group —(C(R^{a})(OH))ₘ— is an integer from 1 to 12;
V¹, V², V³, V⁴, V⁵ and V⁶ are each independently selected from the group consisting of a covalent bond, —C(O)—, —(CH₂)_{q}—C(O)—, —C(O)—(CH₂)_{q}—, —NR¹⁵—,
—NR¹⁵(CH₂)_{q}—, —NR¹⁵(C₆H₄)—, —(CH₂)_{q}—C(O)NR¹⁵—, —C(O)N(R¹⁵)—, —C(O)N(R¹⁵)—(CH₂)_{q}—, —NR¹⁵C(O)—, —(CH₂)_{q}—NR¹⁵C(O)—, —C(O)O—, —(CH₂)_{q}—C(O)O—, —OC(O)—, —OC(O)—(CH₂)_{q}—, —O—, —S—, —S(O)—, —SO₂—, —SO₂N(R¹⁵)—, —(CH₂)_{q}—SO₂N(R¹⁵)—, —NR¹⁵SO₂— and —P(O)OH—, wherein each q is an integer from 1 to 6; and
each R¹⁵ is independently selected from R^{a} and —C(O)R^{a}.
26. The compound of embodiment 23, wherein R² is —X-L^{B}-W.
27. The compound of embodiment 26, wherein -L^{B}- comprises:

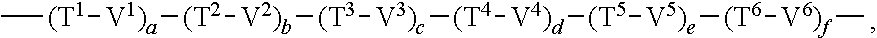

wherein
a, b, c, d, e and f are each independently 0 or 1;
T¹, T², T³, T⁴, T⁵ and T⁶ are each independently selected from a covalent bond, (C₁-C₁₂)alkylene, substituted (C₁-C₁₂)alkylene, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(C(R^{a})(OH))ₘ—, 4-amino-piperidine (4AP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each m is an integer from 1 to 12;
V¹, V², V³, V⁴, V⁵ and V⁶ are each independently selected from the group consisting of a covalent bond, —C(O)—, —(CH₂)_{q}—C(O)—, —C(O)—(CH₂)_{q}—, —NR¹⁵—,
—NR¹⁵(CH₂)_{q}—, —NR¹⁵(C₆H₄)—, —(CH₂)_{q}—C(O)NR¹⁵—, —C(O)N(R¹⁵)—, —C(O)N(R¹⁵)—(CH₂)_{q}—, —NR¹⁵C(O)—, —(CH₂)_{q}—NR¹⁵C(O)—, —C(O)O—, —(CH₂)_{q}—C(O)O—, —OC(O)—, —OC(O)—(CH₂)_{q}—, —O—, —S—, —S(O)—, —SO₂—, —SO₂N(R¹⁵)—, —(CH₂)_{q}—SO₂N(R¹⁵)—, —NR¹⁵SO₂— and —P(O)OH—, wherein each q is an integer from 1 to 6; and
each R¹⁵ is independently selected from R^{a} and —C(O)R^{a}.
28. The compound of any one of embodiments 23-27, wherein the linker L^{A}, L^{B}, or both
   have the formula

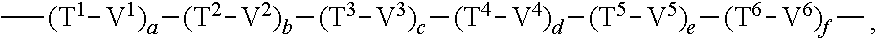

wherein
T¹ is selected from (PEG)ₙ, wherein n is 1 or 2;
V¹ is —NR¹⁵—;
R¹⁵ is hydrogen; and
b, c, d, e and f are each 0.
29. The compound of any one of embodiments 23-27, wherein the linker L^{A}, L^{B}, or both
   have the formula

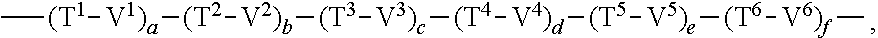

wherein
a and b are each 1, and c, d, e and fare each 0;
T¹ is selected from (PEG)ₙ, wherein n is 1 or 2;
V¹ is —C(O)N(R¹⁵)—;
R¹⁵ is hydrogen;
T² is (C₁-C₁₂)alkyl; and
V² is —O—.
30. The compound of any one of embodiments 23-27, wherein the linker L^{A}, L^{B}, or both
   have the formula

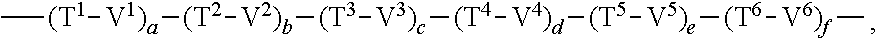

wherein
a is 1, and b, c, d, e and f are each 0;
T¹ is selected from (PEG)ₙ, wherein n is 1 or 2;
V¹ is —NR¹⁵—; and
R¹⁵ is hydrogen.
31. The compound of any one of embodiments 23-27, wherein the linker L^{A}, L^{B}, or both include an alkynylene tether group.
32. The compound of any one of embodiments 23-27, wherein the linker L^{A}, L^{B}, or both include a piperazinyl tether group.
33. he compound of any one of embodiments 23-29, wherein W is a ligand for CRBN, VHL, IAP, MDM2, FEM1B, RNF114, or a combination thereof.
34. The compound of embodiment 33, wherein W is a CRBN ligand.
35. The compound of embodiment 34, wherein the CRBN ligand has a structure selected from:

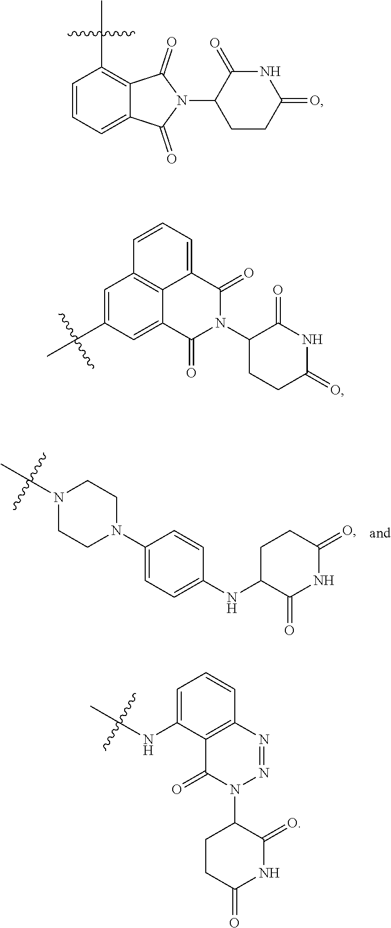 [SMILES: *c1cccc2c1C(=O)N(C1CCC(=O)NC1=O)C2=O ; *c1cc2c3c(cccc3c1)C(=O)N(C1CCC(=O)NC1=O)C2=O ; *N1CCN(c2ccc(NC3CCC(=O)NC3=O)cc2)CC1 ; *Nc1cccc2nnn(C3CCC(=O)NC3=O)c(=O)c12]

36. The compound of embodiment 33, wherein W is a VHL ligand.
37. The compound embodiment 36, wherein the VHL ligand has a structure

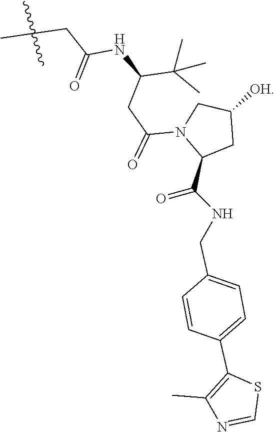 [SMILES: *CC(=O)N[C@H](CC(=O)N1C[C@H](O)C[C@H]1C(=O)NCc1ccc(-c2scnc2C)cc1)C(C)(C)C]

38. The compound of embodiment 33, wherein W is a FEM1B ligand.
39. The compound of embodiment 38, wherein the FEM1B ligand has a structure:

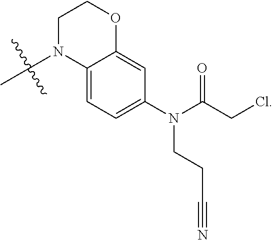 [SMILES: *N1CCOc2cc(N(CCC#N)C(=O)CCl)ccc21]

40. The compound of embodiment 33, wherein W is an RNF114 ligand.
41. The compound of embodiment 40, wherein the RNF114 ligand has a structure:

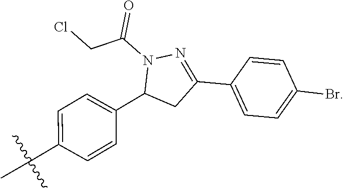 [SMILES: *c1ccc(C2CC(c3ccc(Br)cc3)=NN2C(=O)CCl)cc1]

42. A compound according to any one of embodiments 23-41, having a formula selected from the group consisting of

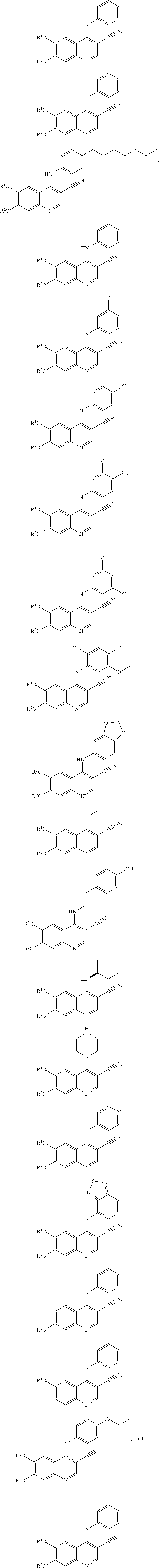

43. The compound of any one of embodiments 23-42 in the form of a pharmaceutically acceptable salt.
44. A pharmaceutical composition comprising an effective amount of a compound of any one of embodiments 23-42.

The following is offered by way of illustration and not by way of limitation.

### EXAMPLES

### Example 1

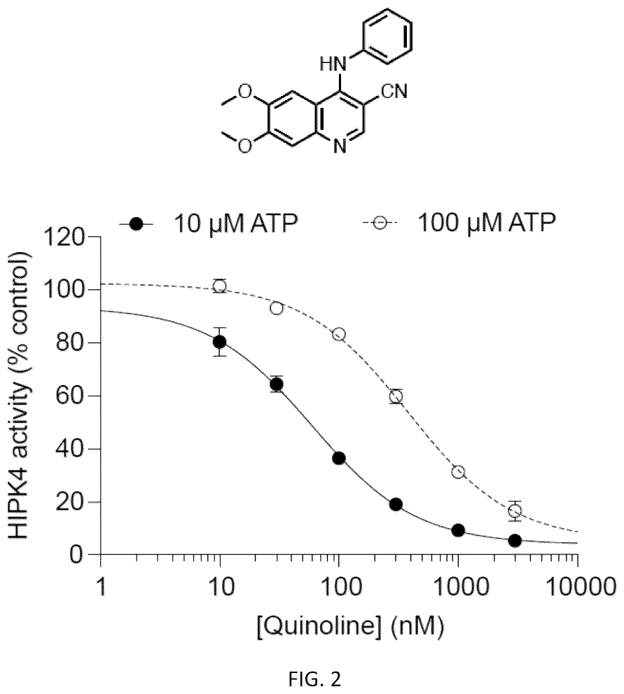

Inhibition of HIP Kinase (HIPK) activity was measured in ADP-Glo assays with myelin basic protein as a universal substrate. FIG. 2 provides dose response curves of the illustrated quinoline compound against HIPK4 at two different ATP concentrations. These data demonstrate inhibition of HIPK4 in an ATP-competitive manner.

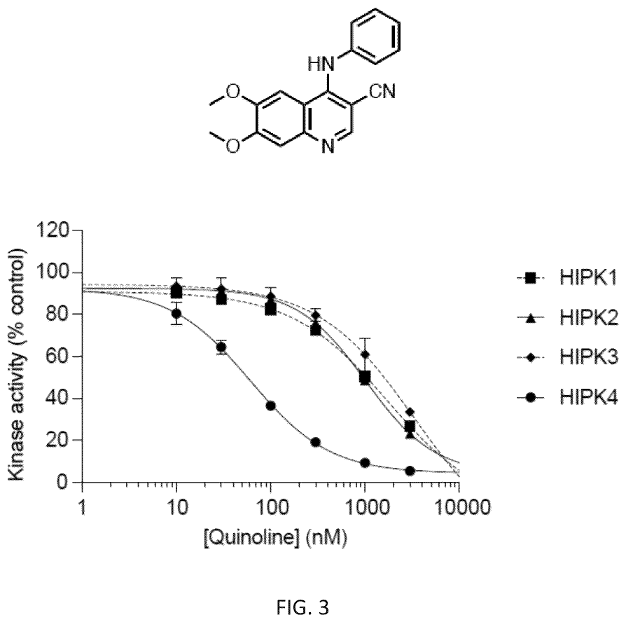

FIG. 3 provides dose-response curves of the illustrated quinoline against each HIPK family member. These data demonstrate selective inhibition of HIPK4 within the HIPK family by the present compounds. HIPK activity was measured in ADP-Glo assays with 10 μM ATP and myelin basic protein as a universal substrate. Data are the average of two biological replicates±s.e.m.

### Example 2

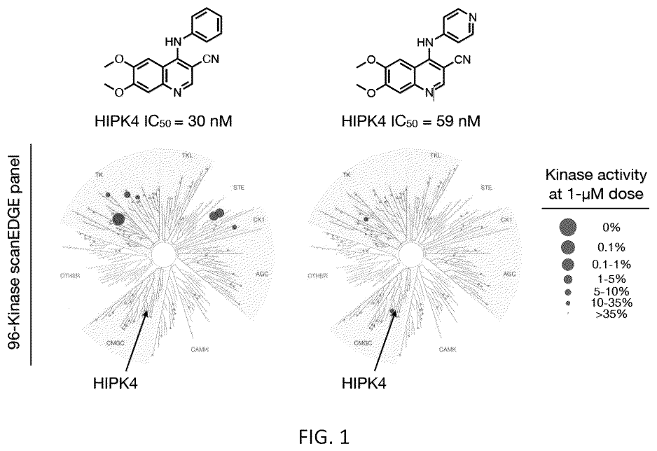

This Example describes the evaluation of target specificity of exemplary compounds for HIPK4 inhibition vis-à-vis other kinases using the Eurofins scanEDGE platform. This screen uses immobilized kinase ligands and an active site-directed competition binding assay, and it includes 97 kinases that are distributed across the major kinase families and clinically relevant variants. The results, illustrated in FIG. 1, show that Compound 1 inhibited the ligand interactions of 11 kinases by at least 65% at a 1-μM dose, whereas Compound 22 inhibited four kinases by at least 65% at 1-μM: DYRK1B (which is closely related to the HIPK family in the kinase phylogenetic tree) and three forms of ABL1.

### Example 3

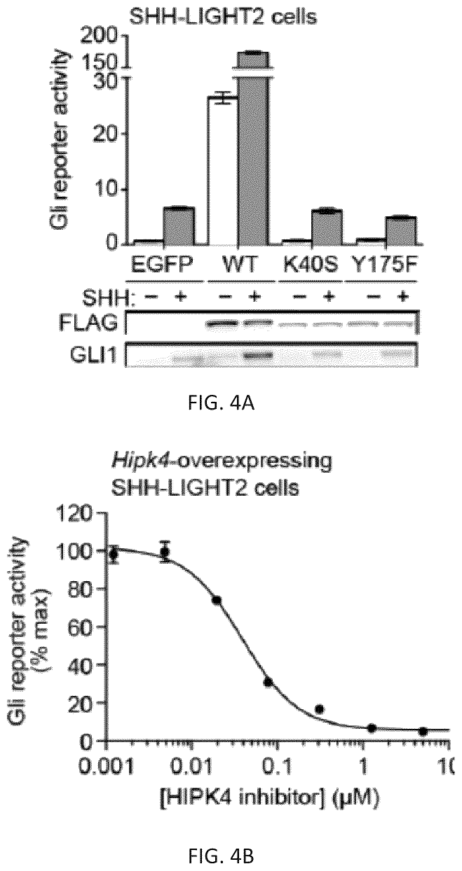

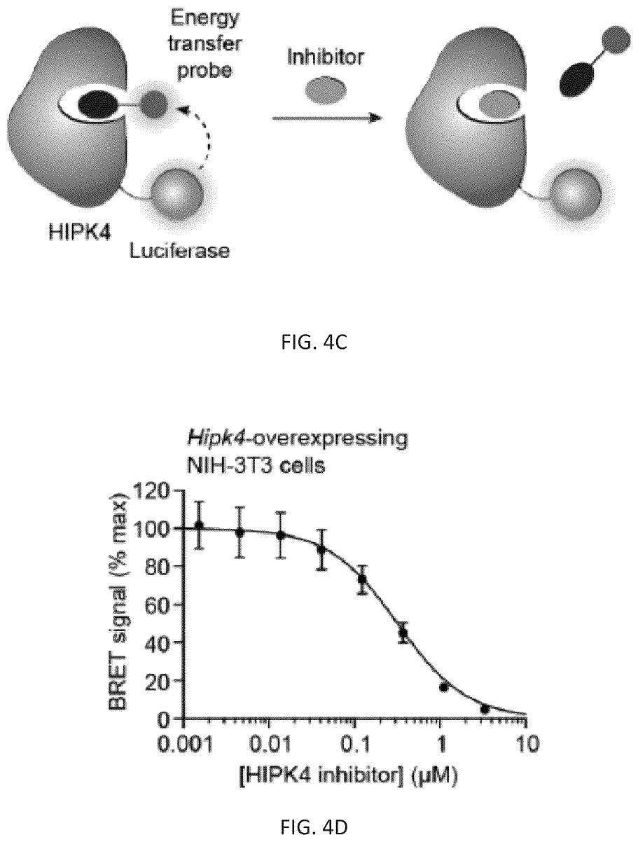

This Example provides a method for quantitatively assessing HIPK4 activity in cells using inhibition of hedgehog signaling as a surrogate marker for HIPK4 inhibition. Since actin regulators have been found to modulate primary cilium function and Hedgehog (Hh)/GLI signaling, we determined whether HIPK4 overexpression affects Gli transcription factor activity. Using NIH-3T3 cells carrying a Gli-dependent firefly luciferase reporter (SHH-LIGHT2 cells)(see, Taipale, J., Chen, J. K., Cooper, M. K., Wang, B., Mann, R. K., Milenkovic, L., Scott, M. P., and Beachy, P. A. (2000) Effects of oncogenic mutations in Smoothened and Patched can be reversed by cyclopamine. Nature 406: 1005-1009), we determined that exogenous Hipk4 can upregulate Gli activity and potentiate Sonic Hedgehog (SHH) signaling. See, FIG. 4A, which provides comparison of wild-type, K40S, and Y175F HIPK4 activities in SHH-LIGHT2 cells as well as Western blots of the FLAG-tagged proteins and Hedgehog pathway target GLI1. Overexpressing inactive HIPK4 mutants (K40S and Y175F) did not significantly affect Gli reporter activity. Moreover, HIPK4-dependent Gli activation could be suppressed by a Type II HIPK4 inhibitor (FIG. 4B). To obtain a more direct measure of HIPK4 engagement in live cells, we implemented a BRET assay developed by Promega (Vasta, J. D. et al. (2018) Quantitative, wide-spectrum kinase profiling in live cells for assessing the effect of cellular ATP on target engagement. Cell Chem. Biol. 25: 206-214 e211). In this method, a HIPK4-NanoLuc luciferase fusion protein is overexpressed in cultured cells, which are then treated with a NanoLuc substrate and an ATP-competitive inhibitor coupled to an energy transfer probe. Binding of the cell-permeable probe to HIPK4 generates a BRET signal that can be measured using a luminometer equipped with spectral filters (FIG. 4C). Competition experiments can be used to quantify small-molecule interactions with the HIPK4 catalytic site (FIGS. 4C-D), and compound cell permeabilities can be assessed by comparing live-cell versus lytic forms of the BRET assay. Graphed data in FIGS. 4B and 4D are the average of three biological replicates±s.e.m.

### Example 4

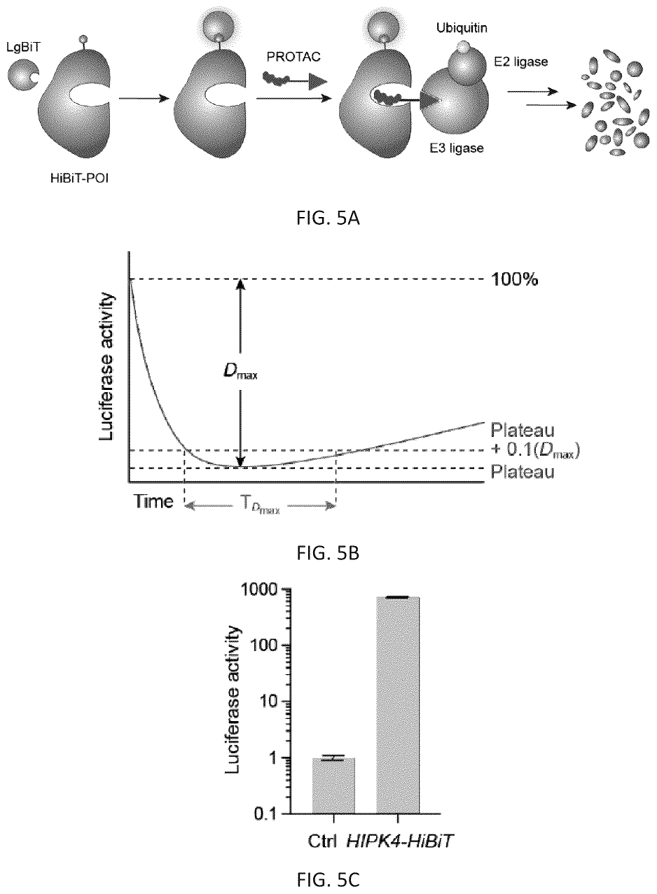

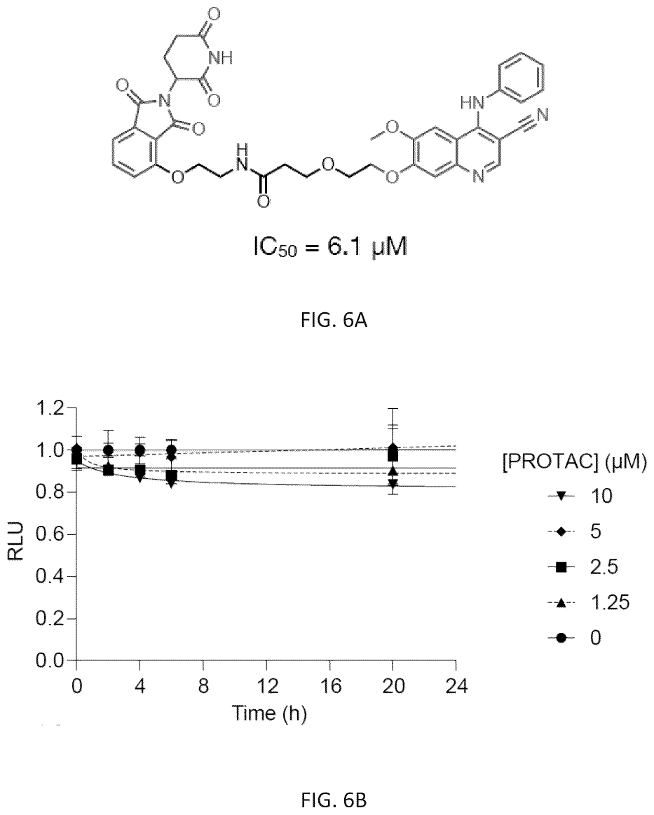

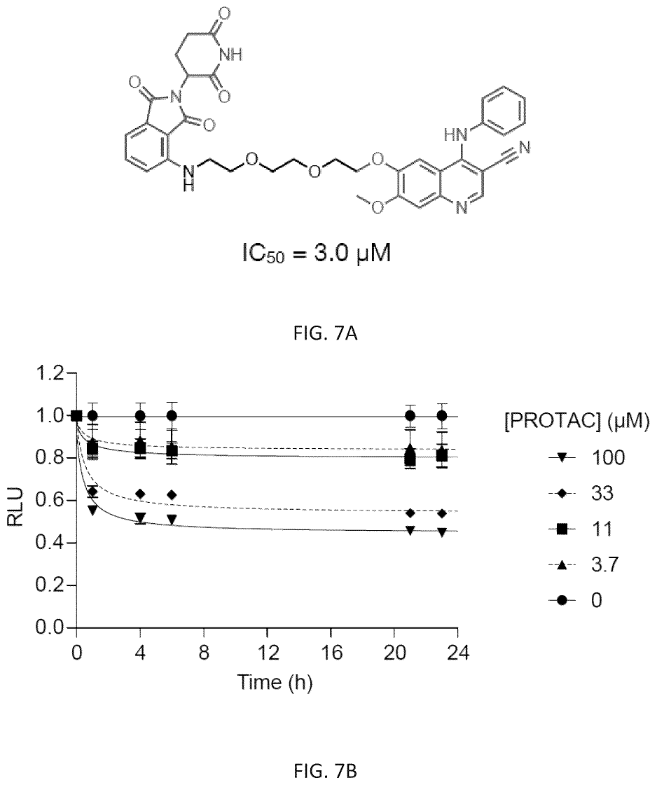

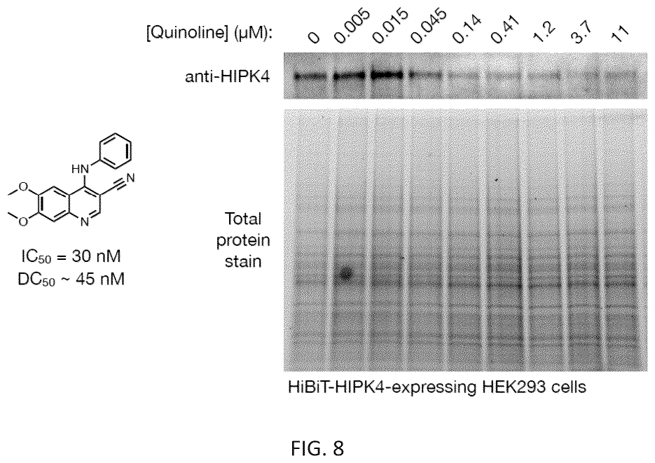

This Example describes quantitative measurements of HIPK4 degradation using split-luciferase system developed by Promega, which utilizes an 11-amino acid peptide (HiBiT) that structurally complements an 18-kDa subunit derived from NanoLuc (LgBiT) to create a functional luciferase (Kd=700 pM) (FIG. 5A) (Riching, K. M., Mahan, S., Corona, C. R., McDougall, M., Vasta, J. D., Robers, M. B., Urh, M., and Daniels, D. L. (2018) Quantitative live-cell kinetic degradation and mechanistic profiling of PROTAC mode of action. ACS Chem. Biol. 13: 2758-2770). With continued reference to FIG. 5A, the protein of interest (POI) is tagged in-frame with the HiBiT peptide and co-expressed with the LgBiT subunit in eukaryotic cells, establishing a cell-based system that enables real-time, quantitative measurements of POI homeostasis. FIG. 5A depicts a PROTAC initiating degradation of the protein of interest, however, surprisingly, as disclosed herein, a small molecule inhibitor, such as a quinoline inhibitor can be used in place of the PROTAC to initiate degradation. In general, protein degradation profiles have three distinct phases: an initial rapid degradation of the target, a degradation plateau, and an eventual slower upward rise due to target recovery (FIG. 5B). Recovery curves are highly variable and multiphasic and therefore not readily quantifiable. However, the first two phases allow the measurement of key parameters such as degradation rates (I), maximal degradation efficacy (Dmax, typically represented as the percent decrease in POI levels), and residence time at Dmax (TDmax). The degradation rate (I) can be calculated from the equation: y=(y₀−Plateau)e^{-λt}+Plateau. Degradation potency (DC₅₀) also can be derived from a plot of Dmax vs. PROTAC concentration (or concentration of small molecule initiator of degradation as disclosed herein).

To comprehensively monitor the HIPK4 degradation profiles of candidate PROTACs and small molecule initiators of degradation, a HIPK4-HiBiT expression construct is transfected into a LgBiT-expressing immortalized cell line that is commercially available from Promega (FIG. 5C). With continued reference to FIG. 5C, data are the average of three biological replicates±s.e.m.

This line has endogenous expression of CRBN and FEM1B, and it has been used to evaluate PROTACs that recruit these E3 ligases (See, for example, Henning, N.J., Manford, A. G., Spradlin, J. N., Brittain, S. M., Zhang, E., McKenna, J. M., Tallarico, J. A., Schirle, M., Rape, M., and Nomura, D. K. (2022) Discovery of a covalent FEM1B recruiter for targeted protein degradation applications. J. Am. Chem. Soc. 144: 701-708). Stable clonal lines are generated and those with the lowest level of HIPK4-HiBiT expression that can be robustly detected through NanoLuc activity are selected. RNF114-dependent protein degradation also has been observed in immortalized cells transfected with an RNF114 expression construct (Yang, Y., Zhou, C., Wang, Y., Liu, W., Liu, C., Wang, L., Liu, Y., Shang, Y., Li, M., Zhou, S., Wang, Y., Zeng, W., Zhou, J., Huo, R., and Li, W. (2017) The E3 ubiquitin ligase RNF114 and TAB1 degradation are required for maternal-to-zygotic transition. EMBO Rep. 18: 205-216). Cell-based tools for assessing RNF114-recruiting PROTACs are generated by stably transfecting exogenous RNF114 into the HIPK4-HiBiT- and LgBiT-expressing cell lines.

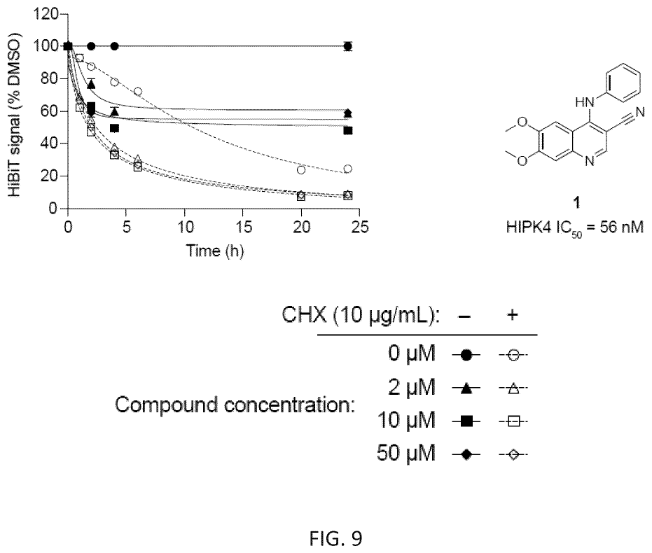

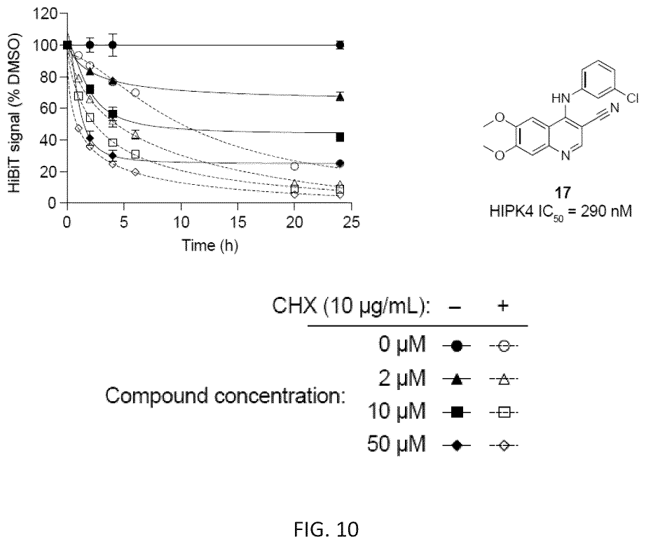

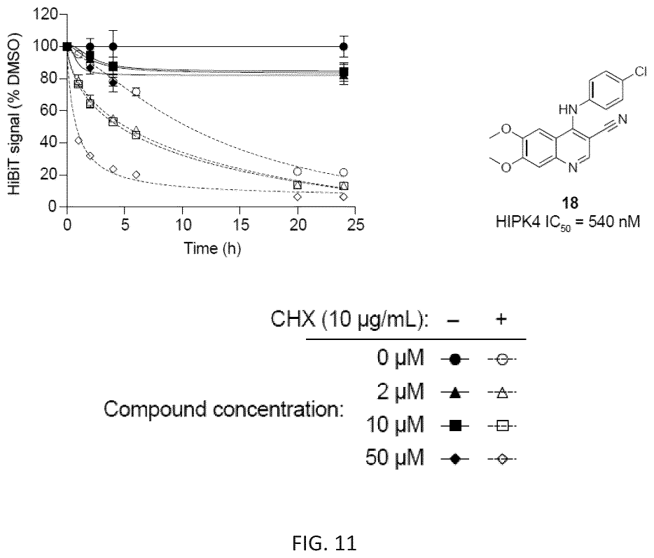

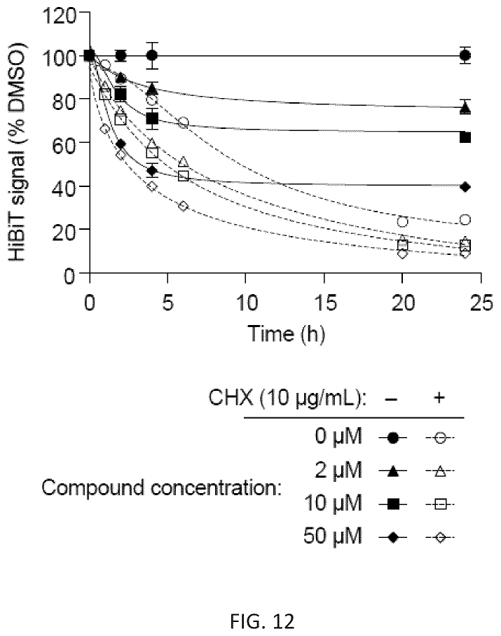

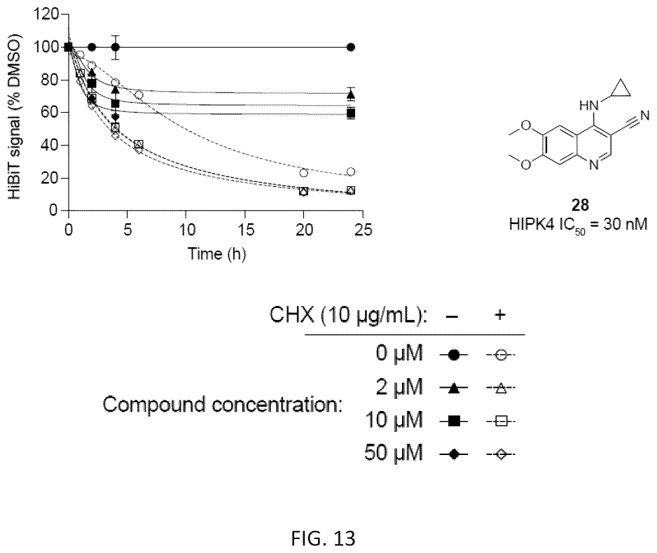

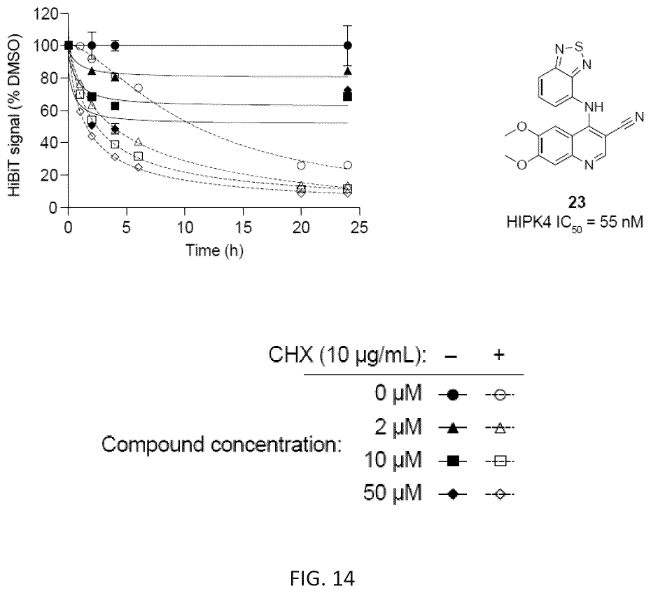

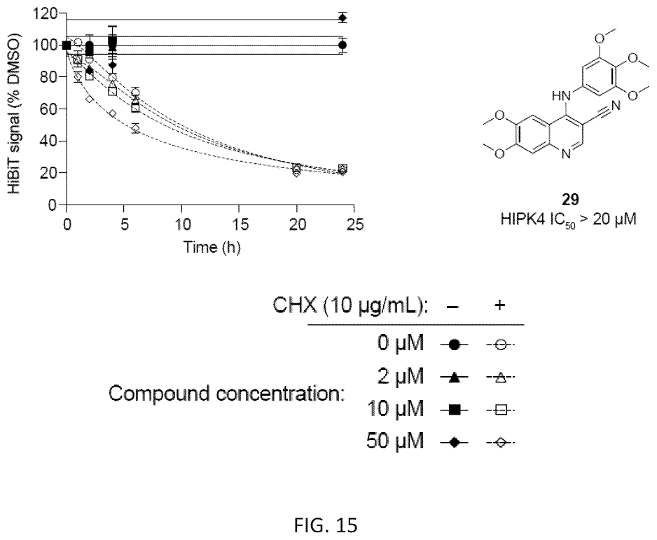

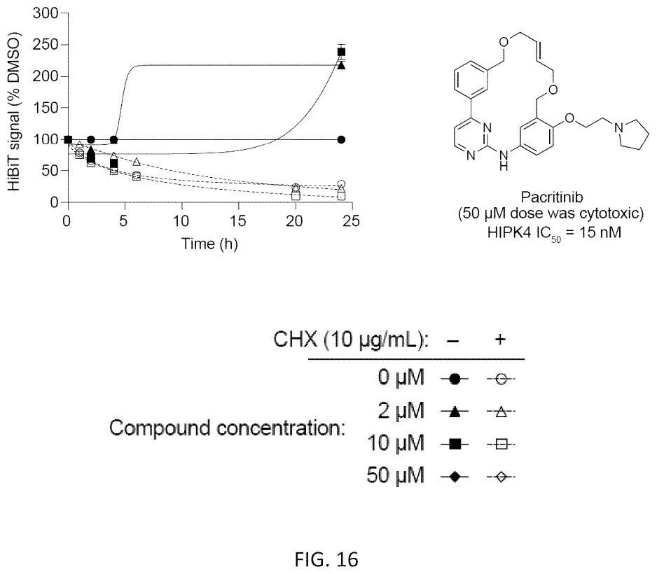

FIGS. 9-15 each provide the chemical structure of a presently disclosed compound and its IC₅₀ against HIPK4 in the ADP-Glo assay. FIG. 16 shows the promiscuous HIPK4 inhibitor pacritinib for comparison. Immortalized cells stably expressing HiBiT-HIPK4 and LgBiT constructs cells were treated varying concentrations of the illustrated compounds in the presence or absence of cycloheximide (indicated as “−” or “+”), and cell luciferase activities (RLU) were continually monitored for 24 hours using the stabilized furimazine substrate Endurazine. The results graphed in FIGS. 9-16 along with a graph showing the quantitative measurement of degradation activity by the present compounds in immortalized cells stably expressing HiBiT-HIPK4 and LgBiT constructs, as demonstrated by decreasing HiBiT signal versus time. Data are the average RLU values of three biological replicates, normalized to DMSO-treated control cells.

### Example 5

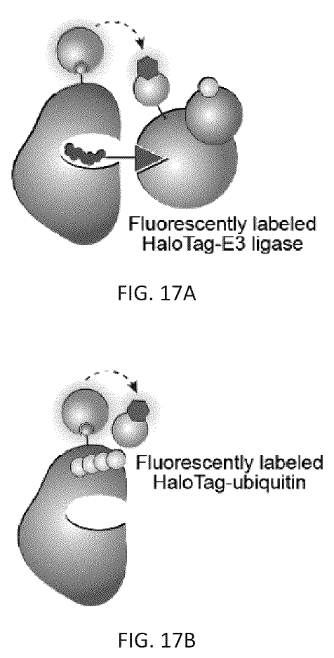

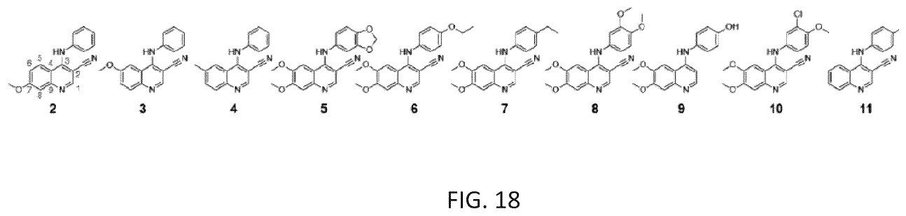

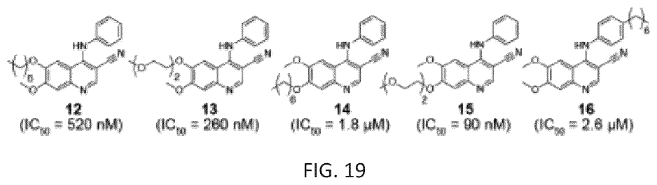

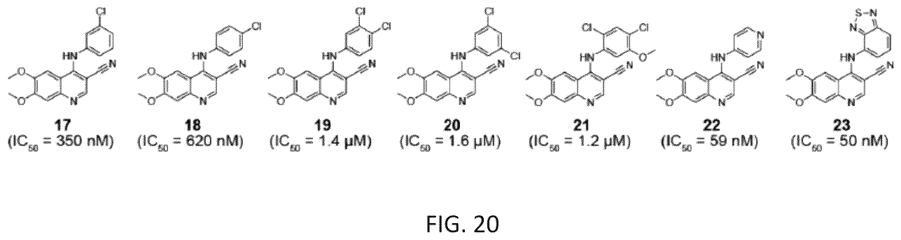

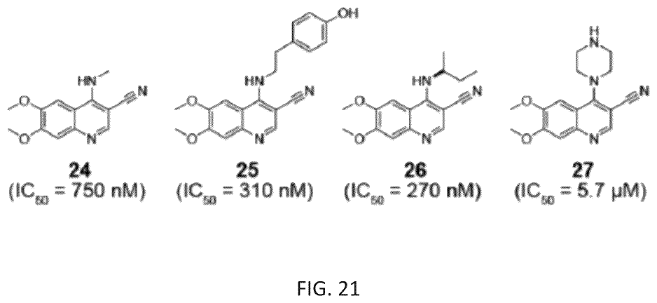

This Example describes quantitative measurement of PROTAC-mediated ternary complex formation and ubiquitination. HIPK4-HiBiT- and LgBiT-expressing cell lines are used to generate cell-based assays of steps that lead to PROTAC-mediated HIPK4 degradation, namely ternary complex formation and HIPK4 degradation. These assays rely on E3 ligase and ubiquitin constructs that are fused to HaloTag, a 33-kDa genetically modified bacterial haloalkane dehalogenase that can be bioorthogonally labeled in live cells with chloroalkane-functionalized fluorescent dyes. With reference to FIG. 17A, ternary complex formation is confirmed by transfecting HIPK4-HiBiT- and LgBiT-expressing cell lines with plasmid vectors encoding CRBN, VHL, IAP, MDM2, FEM1B, or RNF114 with an N-terminal HaloTag. The tagged E3 ligases are then covalently labeled in situwith a commercial cell-permeable red fluorescent dye with a chloroalkane linker. When the resulting cells are cultured with a NanoLuc substrate, PROTAC-mediated recruitment of HIPK4 and the HaloTag-E3 ligase generates a BRET signal that is measured as a ratio of 618-nm fluorescence over 460-nm luminescence. In an analogous manner, polyubiquitination of HIPK4 (FIG. 17B) can be monitored in real time by transfecting the HIPK4-HiBiT- and LgBiT-expressing cell lines with a commercial vector encoding a HaloTag-ubiquitin fusion protein and treating the lines with the chloroalkyl fluorophore.

### Example 6

This Example describes methods for synthesizing embodiments of the presently disclosed compounds. Scheme (I) illustrates introduction of an amino group at the 4-position of quinoline starting material, Compound I, to give 4-amino quinoline derivative III. Compound I (1 mmol) and compound II (1.1 mmol) are suspended in ethanol (5 mL). The mixture is refluxed at 100° C. for 18 hours. The solution is concentrated in vacuo, and the residue is purified by silica gel chromatography using ethyl acetate/hexane followed by 1-5% methanol in ethyl acetate to give Compound III.

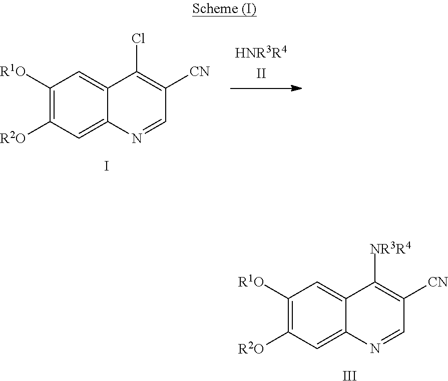

Scheme (II) illustrates deblocking of the 6-position phenol of intermediate Compound IV.

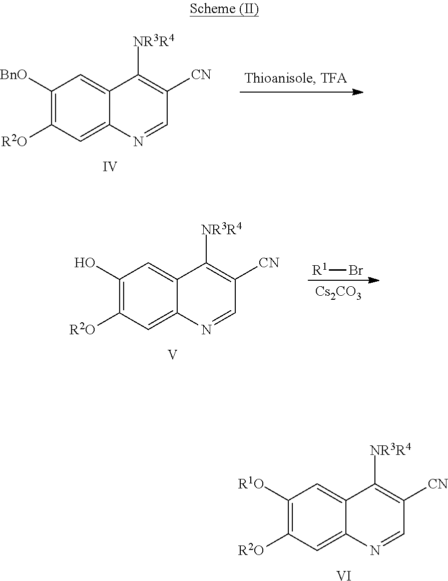

With reference to Scheme (II), Compound IV (1 mmol), thioanisole (20 mmol), and TFA (50 mmol) are mixed, and stirred at 80° C. for 2 hours. Then the solution is concentrated in vacuo, and the residue is resuspended with ice water. NH₄OH is slowly added into the solution mixture, and then filtered. The solids are washed with ethyl acetate to give Compound V, which is redissolved with DMF. Cesium carbonate (1 mmol) is added, followed by stirring at room temperature for 30 minutes. Then R¹—Br is added, and the mixture is refluxed at 100° C. for 2 hours. The reaction is quenched with water, and the product is extracted with ethyl acetate. The organic layer is dried over anhydrous sodium sulfate, filtered, and concentrated in vacuo. The residue is purified by silica gel chromatography (50% ethyl acetate in hexane) to give Compound VI.

Scheme (III) illustrates the introduction of a substituent at the 7-position phenol.

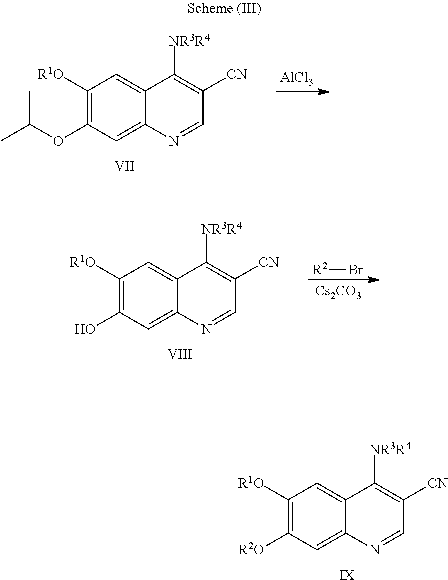

With reference to Scheme (III), Compound VII (1 mmol) is dissolved in anhydrous dichloromethane, and followed by addition of aluminum chloride (1.4 mmol). The mixture is stirred at 0° C. for 2 hours, and then filtered. The solids are washed with HCl (0.1 N) and water to give Compound VIII, which is redissolved with DMF. Cesium carbonate (1 mmol) is added, followed by stirring at room temperature for 30 minutes. Then R²—Br is added, and the mixture is refluxed at 100° C. for 2 hours. The reaction is quenched with water, and the product is extracted with ethyl acetate. The organic layer is dried over anhydrous sodium sulfate, filtered, and concentrated in vacuo. The residue is purified by silica gel chromatography (50% ethyl acetate in hexane) to give Compound VI.

Exemplary compounds disclosed herein were prepared consistent with the procedures set forth in Schemes (I), (II) and (III).

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) is not invoked.

What is claimed is:

1. A method for inhibiting HIPK4, comprising contacting the HIPK4 with a compound of the formula: 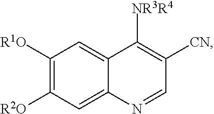
or a pharmaceutically acceptable salt thereof,
wherein
R¹ is independently selected from hydrogen, —X-L^{A}-W, and C₁₋₆ alkyl optionally substituted with one or more R^{a}, R^{b}, or both;
R² is independently selected from hydrogen, —X-L^{B}-W, and C₁₋₆ alkyl optionally substituted with one or more of the same or different R^{a}, R^{b}, or both;
X is, for each occurrence, independently selected from a covalent bond,
—C(O)—, —C(O)O—, —C(O)N(R^{d})—, and —S(O)₂—;
L^{A} is a linker;
L^{B} is a linker;
W is a E3 ubiquitin ligase binding moiety;
R³ and R⁴ are independently selected from hydrogen, C₁₋₆ alkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl, each optionally substituted with one or more of the same or different R^{a}, R^{b}, or both, or R³ and R⁴ are taken together with the nitrogen atom to which they are bonded to form a 5 to 8-membered cycloheteroalkyl or heteroaryl which may optionally include one or more of the same or different additional heteroatoms and which may optionally be substituted with one or more of the same or different which may optionally be substituted with one or more of the same or different R^{e} groups;
each R^{a} is independently selected from hydrogen, C₁₋₈ alkyl, C₂₋₆ alkenyl, C₂₋₆ alkynyl, C₁₋₆ haloalkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl;
R^{b} is independently selected from the group consisting of ═O, —OR^{a}, halogen, C₁₋₃haloalkyloxy, —OCF₃, ═S, —SR^{d}, ═NR^{d}, ═NOR^{d}, —NR^{c}R^{c}, —SF₅, halogen, —CF₃, —CN, —NO₂, —S(O)R^{d}, —S(O)₂R^{d}, —S(O)₂OR^{d},
—N(H)S(O)₂R^{d}, —S(O)NR^{c}R^{c}, —S(O)₂NR^{c}R^{c}, —OS(O)R^{d}, —OS(O)₂R^{d},
—OS(O)₂OR^{d}, —OS(O)₂NR^{c}R^{c}, —C(O)R^{d}, —C(O)OR^{d}, —C(O)NR^{c}R^{c},
—C(NH)NR^{c}R^{c}, —C(NR^{a})NR^{c}R^{c}, —C(NOH)R^{a}, —C(NOH)NR^{c}R^{c}, —OC(O)R^{d},
—OC(O)OR^{d},
—OC(O)NR^{c}R^{c}, —OC(NH)NR^{c}R^{c}, —OC(NR^{a})NR^{c}R^{c}, —[NHC(O)]ₙR^{d},
—[NR^{a}C(O)]ₙR^{d}, —[NHC(O)]ₙOR^{d}, —[NR^{a}C(O)]ₙOR^{d}, —[NHC(O)]ₙNR^{c}R^{c},
—[NR^{a}C(O)]ₙNR^{c}R^{c},
—(CHR^{a}CHR^{a}—O)ₙ—R^{a}, —[NHC(NH)]ₙNR^{c}R^{c} and —[NR^{a}C(NR^{a})]ₙNR^{c}R^{c};
each R^{c} independently is hydrogen, R^{a}, or, alternatively, two R^{c} are taken together with the nitrogen atom to which they are bonded to form a 5 to 10-membered heterocyclylalkyl which may optionally include 1, 2 or 3 additional heteroatoms selected from O, N and S, and may optionally be substituted with one or more of the same or different R^{e} groups;
each R^{d} is independently hydrogen or C₁₋₆ alkyl;
each R^{e} is independently R^{d}, ═O, —OR^{a}, halogen, —C(O)R^{d}, and —S(O)₂R^{d}; and
each n is independently an integer from 0 to 3.

2. The method of claim 1, wherein R¹ is C₁₋₆ alkyl optionally substituted with one or more R^{a}, R^{b}, or both.

3. The method of claim 1, wherein R² is C₁₋₆ alkyl optionally substituted with one or more of the same or different R^{a}, R^{b}, or both.

4. The method of claim 1, wherein R¹ is -L^{A}-W.

5. The method of claim 4, wherein L^{A} comprises: 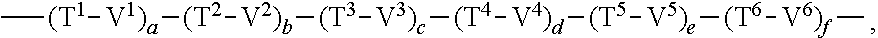
wherein
a, b, c, d, e and f are each independently 0 or 1;
T¹, T², T³, T⁴, T⁵ and T⁶ are each independently selected from a covalent bond, (C₁-C₁₂)alkylene, substituted (C₁-C₁₂)alkylene, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(C(R^{a})(OH))ₘ—, 4-amino-piperidine (4AP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n in the group (PEG)ₙ is an integer from 1 to 30, each p is an integer from 1 to 20, and each m in the group —(C(R^{a})(OH))ₘ— is an integer from 1 to 12;
V¹, V², V³, V⁴, V⁵ and V⁶ are each independently selected from the group consisting of a covalent bond, —C(O)—, —(CH₂)_{q}—C(O)—, —C(O)—(CH₂)_{q}—, —NR¹⁵—,
—NR¹⁵(CH₂)_{q}—, —NR¹⁵(C₆H₄)—, —(CH₂)_{q}—C(O)NR¹⁵—, —C(O)N(R¹⁵)—, —C(O)N(R¹⁵)—(CH₂)_{q}—, —NR¹⁵C(O)—, —(CH₂)_{q}—NR¹⁵C(O)—, —C(O)O—, —(CH₂)_{q}—C(O)O—, —OC(O)—, —OC(O)—(CH₂)_{q}—, —O—, —S—, —S(O)—, —SO₂—, —SO₂N(R¹⁵)—, —(CH₂)_{q}—SO₂N(R¹⁵)—, —NR¹⁵SO₂— and —P(O)OH—, wherein each q is an integer from 1 to 6; and
each R¹⁵ is independently selected from R^{a} and —C(O)R^{a}.

6. The method of claim 1, wherein R² is -L^{B}-W.

7. The method of claim 6, wherein -L^{B}- comprises: 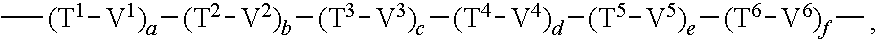
wherein
a, b, c, d, e and f are each independently 0 or 1;
T¹, T², T³, T⁴, T⁵ and T⁶ are each independently selected from a covalent bond, (C₁-C₁₂)alkylene, substituted (C₁-C₁₂)alkylene, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(C(R^{a})(OH))ₘ—, 4-amino-piperidine (4AP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each m is an integer from 1 to 12;
V¹, V², V³, V⁴, V⁵ and V⁶ are each independently selected from the group consisting of a covalent bond, —C(O)—, —(CH₂)_{q}—C(O)—, —C(O)—(CH₂)_{q}—, —NR¹⁵—,
—NR¹⁵(CH₂)_{q}—, —NR¹⁵(C₆H₄)—, —(CH₂)_{q}—C(O)NR¹⁵—, —C(O)N(R¹⁵)—, —C(O)N(R¹⁵)—(CH₂)_{q}—, —NR¹⁵C(O)—, —(CH₂)_{q}—NR¹⁵C(O)—, —C(O)O—, —(CH₂)_{q}—C(O)O—, —OC(O)—, —OC(O)—(CH₂)_{q}—, —O—, —S—, —S(O)—, —SO₂—, —SO₂N(R¹⁵)—, —(CH₂)_{q}—SO₂N(R¹⁵)—, —NR¹⁵SO₂— and —P(O)OH—, wherein each q is an integer from 1 to 6; and
each R¹⁵ is independently selected from R^{a} and —C(O)R^{a}.

8. The method of claim 7, wherein W is a ligand for CRBN, VHL, IAP, MDM2, FEM1B, RNF114, or a combination thereof.

9. The method of claim 8, wherein W has a structure selected from:

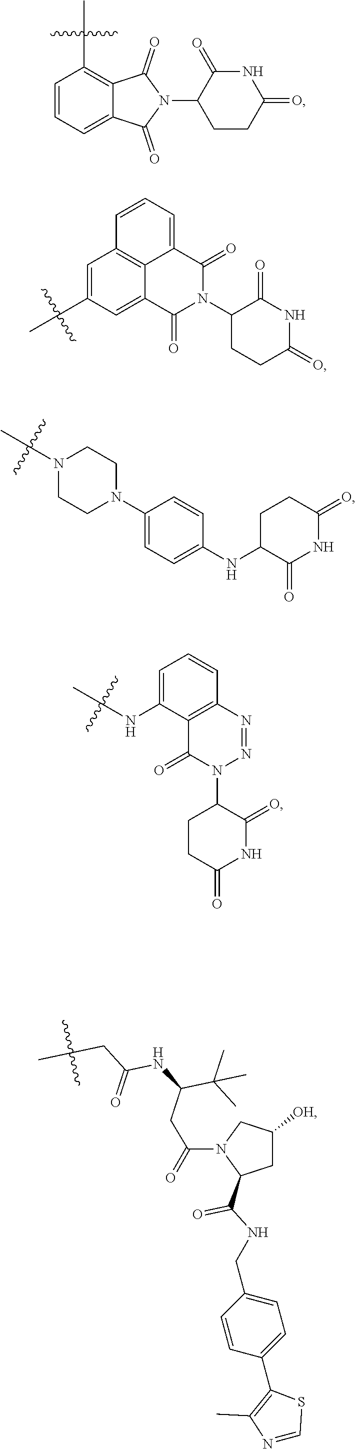

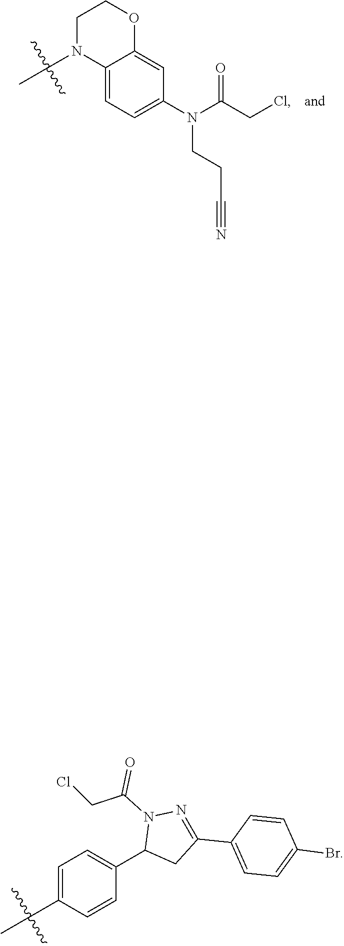

10. The method of claim 1, wherein the compound has the formula

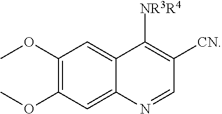

11. The method of claim 1, wherein one of R³ and R⁴ is selected from C₁₋₆ alkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl, each optionally substituted with one or more of the same or different R^{a}, R^{b}; and the other of R³ and R⁴ is hydrogen.

12. The method of claim 1 wherein contacting the HIPK4 induces HIPK4 degradation.

13. The method of claim 1, wherein the compound has the formula

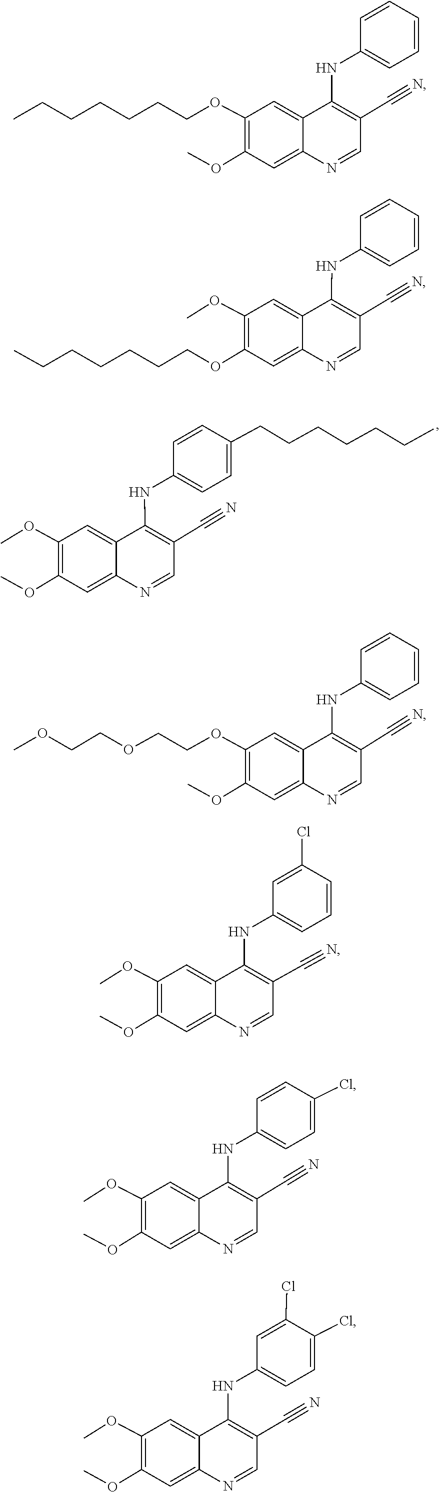

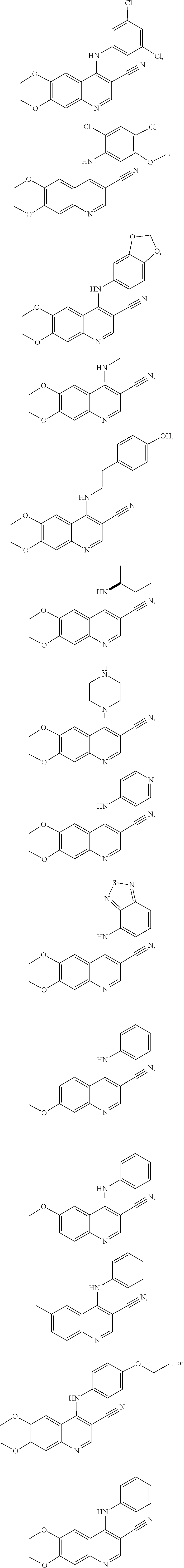

14. A method for inhibiting spermiogenesis, comprising administering an effective amount of a compound according to the formula: 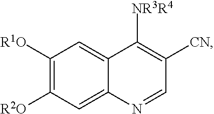
wherein
R¹ is independently selected from hydrogen, —X-L^{A}-W, and C₁₋₆ alkyl optionally substituted with one or more R^{a}, R^{b}, or both;
R² is independently selected from hydrogen, —X-L^{B}-W, and C₁₋₆ alkyl optionally substituted with one or more of the same or different R^{a}, R^{b}, or both;
X is, for each occurrence, independently selected from a covalent bond,
—C(O)—, —C(O)O—, —C(O)N(R^{d})—, and —S(O)₂—;
L^{A} is a linker;
L^{B} is a linker;
W is a E3 ubiquitin ligase binding moiety;
R³ and R⁴ are independently selected from hydrogen, C₁₋₆ alkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl, each optionally substituted with one or more of the same or different R^{a}, R^{b}, or both, or R³ and R⁴ are taken together with the nitrogen atom to which they are bonded to form a 5 to 8-membered cycloheteroalkyl or heteroaryl which may optionally include one or more of the same or different additional heteroatoms and which may optionally be substituted with one or more of the same or different which may optionally be substituted with one or more of the same or different R^{e} groups;
each R^{a} is independently selected from hydrogen, C₁₋₈ alkyl, C₂₋₆ alkenyl, C₂₋₆ alkynyl, C₁₋₆ haloalkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl;
R^{b} is independently selected from the group consisting of ═O, —OR^{a}, halogen, C₁₋₃haloalkyloxy, —OCF₃, ═S, —SR^{d}, ═NR^{d}, ═NOR^{d}, —NR^{c}R^{c}, —SF₅, halogen, —CF₃, —CN, —NO₂, —S(O)R^{d}, —S(O)₂R^{d}, —S(O)₂OR^{d},
—N(H)S(O)₂R^{d}, —S(O)NR^{c}R^{c}, —S(O)₂NR^{c}R^{c}, —OS(O)R^{d}, —OS(O)₂R^{d},
—OS(O)₂OR^{d}, —OS(O)₂NR^{c}R^{c}, —C(O)R^{d}, —C(O)OR^{d}, —C(O)NR^{c}R^{c},
—C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d},
—OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —(CHR^{a}CH R^{a}—O)ₙ—R^{a}, —[NHC(NH)]ₙNR^{c}R^{c} and —[NR^{a}C(NR^{a})]ₙNR^{c}R^{c};
each R^{c} independently is hydrogen, R^{a}, or, alternatively, two R^{c} are taken together with the nitrogen atom to which they are bonded to form a 5 to 10-membered heterocyclylalkyl which may optionally include 1, 2 or 3 additional heteroatoms selected from O, N and S, and may optionally be substituted with one or more of the same or different R^{e} groups;
each R^{d} is independently hydrogen or C₁₋₆ alkyl;
each R^{e} is independently R^{d}, ═O, —OR^{a}, halogen, —C(O)R^{d}, and —S(O)₂R^{d}; and
each n is independently an integer from 0 to 3.

15. A compound of the formula: 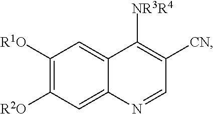
or a pharmaceutically acceptable salt thereof,
wherein
R¹ is independently selected from hydrogen, —X-L^{A}-W, and C₁₋₆ alkyl optionally substituted with one or more R^{a}, R^{b}, or both;
R² is independently selected from hydrogen, —X-L^{B}-W, and C₁₋₆ alkyl optionally substituted with one or more of the same or different R^{a}, R^{b}, or both; provided that when R¹ is hydrogen or C₁₋₆ alkyl optionally substituted with one or more R^{a}, R^{b}, or both, R² is —X-L^{B}-W, and when R² is hydrogen or C₁₋₆ alkyl optionally substituted with one or more R^{a}, R^{b}, or both, R¹ is —X-L^{A}-W;
X is, for each occurrence, independently selected from a covalent bond,
—C(O)—, —C(O)O—, —C(O)N(R^{d})—, and —S(O)₂—;
L^{A} is a linker;
L^{B} is a linker;
W is a E3 ubiquitin ligase binding moiety;
R³ and R⁴ are independently selected from hydrogen, C₁₋₆ alkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl, each optionally substituted with one or more of the same or different R^{a}, R^{b}, or both, or R³ and R⁴ are taken together with the nitrogen atom to which they are bonded to form a 5 to 8-membered cycloheteroalkyl or heteroaryl which may optionally include one or more of the same or different additional heteroatoms and which may optionally be substituted with one or more of the same or different which may optionally be substituted with one or more of the same or different R^{e} groups;
each R^{a} is independently selected from hydrogen, C₁₋₈ alkyl, C₂₋₆ alkenyl, C₂₋₆ alkynyl, C₁₋₆ haloalkyl, C₃₋₈ cycloalkyl, C₆₋₁₀ aryl, C₅₋₁₀ heteroaryl, C₆₋₁₆ arylalkyl, 2-6 membered heteroalkyl and 3-8 membered heterocyclylalkyl;
R^{b} is independently selected from the group consisting of ═O, —OR^{a}, halogen, C₁₋₃haloalkyloxy, —OCF₃, ═S, —SR^{d}, ═NR^{d}, ═NOR^{d}, —NR^{c}R^{c}, —SF₅, halogen, —CF₃, —CN, —NO₂, —S(O)R^{d}, —S(O)₂R^{d}, —S(O)₂OR^{d},
—N(H)S(O)₂R^{d}, —S(O)NR^{c}R^{c}, —S(O)₂NR^{c}R^{c}, —OS(O)R^{d}, —OS(O)₂R^{d}, —OS(O)₂OR^{d},
—OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —(CHR^{a}CHR^{a}—O)ₙ—R^{a}, —[NHC(NH)]ₙNR^{c}R^{c} and —[NR^{a}C(NR^{a})]ₙNR^{c}R^{c};
each R^{c} independently is hydrogen, R^{a}, or, alternatively, two R^{c} are taken together with the nitrogen atom to which they are bonded to form a 5 to 10-membered heterocyclylalkyl which may optionally include 1, 2 or 3 additional heteroatoms selected from O, N and S, and may optionally be substituted with one or more of the same or different R^{e} groups;
each R^{d} is independently hydrogen or C₁₋₆ alkyl;
each R^{e} is independently R^{d}, ═O, —OR^{a}, halogen, —C(O)R^{d}, and —S(O)₂R^{d}; and
each n is independently an integer from 0 to 3.

16. The compound of claim 15, wherein R¹ is —X-L^{A}-W or R² is —X-L^{B}-W.

17. The compound of claim 16, wherein L^{A} and L^{B}, if present comprise: 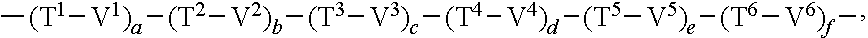
wherein
a, b, c, d, e and f are each independently 0 or 1;
T¹, T², T³, T⁴, T⁵ and T⁶ are each independently selected from a covalent bond, (C₁-C₁₂)alkylene, substituted (C₁-C₁₂)alkylene, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(C(R^{a})(OH))ₘ—, 4-amino-piperidine (4AP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n in the group (PEG)ₙ is an integer from 1 to 30, each p is an integer from 1 to 20, and each m in the group —(C(R^{a})(OH))ₘ— is an integer from 1 to 12;
V¹, V², V³, V⁴, V⁵ and V⁶ are each independently selected from the group consisting of a covalent bond, —C(O)—, —(CH₂)_{q}—C(O)—, —C(O)—(CH₂)_{q}—, —NR¹⁵—,
—NR¹⁵(CH₂)_{q}—, —NR¹⁵(C₆H₄)—, —(CH₂)_{q}—C(O)NR¹⁵—, —C(O)N(R¹⁵)—, —C(O)N(R¹⁵)—(CH₂)_{q}—, —NR¹⁵C(O)—, —(CH₂)_{q}—NR¹⁵C(O)—, —C(O)O—, —(CH₂)_{q}—C(O)O—, —OC(O)—, —OC(O)—(CH₂)_{q}—, —O—, —S—, —S(O)—, —SO₂—, —SO₂N(R¹⁵)—, —(CH₂)_{q}—SO₂N(R¹⁵)—, —NR¹⁵SO₂— and —P(O)OH—, wherein each q is an integer from 1 to 6; and
each R¹⁵ is independently selected from R^{a} and —C(O)R^{a}.

18. The compound of claim 15, wherein W is a ligand for CRBN, VHL, IAP, MDM2, FEM1B, RNF114, or a combination thereof.

19. A compound according to claim 15, having a formula selected from the group consisting of

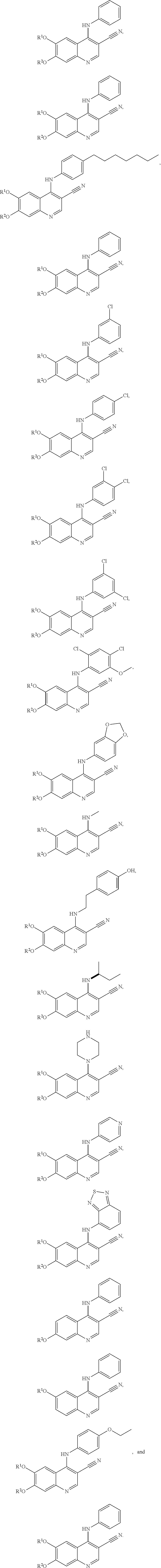

20. A pharmaceutical composition comprising an effective amount of a compound claim 15.
